(19) United States
(12) Patent Application Publication
(10) Pub. No.: US 2013/0273573 A1
(43) Pub. Date: Oct. 17, 2013

(54) AMYLOID BETA AGGREGATES IN CEREBRO SPINAL FLUID AS BIOMARKERS FOR ALZHEIMER'S DISEASE

(71) Applicants: Sophie Allauzen, Emeryville, CA (US); Joseph Fedynyshyn, Emeryville, CA (US); David Peretz, Emeryville, CA (US); Xuemei Wang, Emeryville, CA (US); Alice Yam, Emeryville, CA (US)

(72) Inventors: Sophie Allauzen, Emeryville, CA (US); Joseph Fedynyshyn, Emeryville, CA (US); Carol Man Gao, Emeryville, CA (US); David Peretz, Emeryville, CA (US); Xuemei Wang, Emeryville, CA (US); Alice Yam, Emeryville, CA (US)

(73) Assignee: NOVARTIS AG, Basel (CH)

(21) Appl. No.: 13/512,858

(22) PCT Filed: Nov. 30, 2010
(86) PCT No.: PCT/US2010/058450

§ 371 Date: Nov. 9, 2012

Related U.S. Application Data
(60) Provisional application No. 61/265,340, filed on Nov. 30, 2009.

(51) Int. Cl. G01N 33/543

(52) U.S. Cl. CPC G01N 33/543
USPC 435/7.92; 436/501; 435/7.1

(57) ABSTRACT

The invention provides methods for assessing increased probability of having Alzheimer's disease in a subject under assessment including a step of obtaining a measurement of Aβ40 aggregates in a biological sample from the subject, where the biological sample does not include brain tissue, a fraction of brain tissue, or brain homogenate. In some embodiments, the invention relates to methods of assessing increased probability of early stage Alzheimer's disease. The invention further provides methods of assessing increased probability of not having Alzheimer's disease, methods of monitoring disease progression in a subject with Alzheimer's disease, and methods of assigning disease stage for a subject with Alzheimer's disease, and methods of assessing increased probability of MCI progressing to Alzheimer's disease.

### CROSS REFERENCE TO RELATED APPLICATIONS

This application claims priority to U.S. Prov. App. Ser. No. 61/265,340, filed Nov. 30, 2009, which is hereby incorporated by reference in its entirety.

### BACKGROUND

Alzheimer's disease, the most common form of dementia associated with aging, threatens to become an epidemic over the next 50 years as a high proportion of the world's population grows to be older than 65. Although caring for Alzheimer's patients presents a significant and growing economic burden to society, the few treatments that exist only palliate symptoms of the disease; no preventative treatments are available. Furthermore, current diagnostic methods are dependent on easily observable symptoms of the disease that are apparent only after disease progression is underway.

Alzheimer's disease has been associated with the accumulation of misfolded Aβ and tau proteins in the brain. Detection of the aggregates of these misfolded proteins in living subjects and samples obtained from living subjects has proven difficult. The current techniques for confirming the presence of aggregates in living patients are crude and invasive. For example, histopathological examination would require biopsies that are risky to the subject. Histopathology is inherently prone to sampling error as lesions and deposits of aggregated pathogenic proteins can be missed depending on the area where the biopsy is taken. Thus, definitive diagnosis and palliative treatments for these conditions before death of the subject remains a substantially unmet challenge.

Deposition of amyloid-beta protein (Aβ) aggregates, mainly Aβ 1-40 (Aβ40) and 1-42 (Aβ42), in brain has been linked to Alzheimer's disease (AD) and is considered to be the gold-standard marker for the disease. However, the only definitive test for AD is immunohistochemical staining of plaques of fibrillar Aβ aggregate from post-mortem brain samples. Currently, there are no FDA-approved ante-mortem diagnostic tests for AD. Plasma or CSF samples could be used for ante-mortem tests. Some ante-mortem AD tests have focused on the cerebrospinal fluid (CSF) and attempt to quantitate soluble monomeric Aβ42. However, this biomarker only serves as an indirect measurement of AD.

Recent literature has suggested that small, soluble, non-fibrillar oligomeric species of Aβ are likely to be the neurotoxic agents directly contributing to the Alzheimer's disease phenotype (Hoshi et al., PNAS, 2003, 100, 6370; Lambert et al., PNAS, 1998, 95, 6448; Sakano and Zako, FEBBS J., 2010, 277(6), 1348; Fukumoto et al., 2010, FASEB J., 24, 2716). Furthermore, using antibodies raised against Aβ42 or a seeded multimerization strategy using fluorescently labeled Aβ42, elevated levels of Aβ oligomeric species have been found in cerebrospinal fluid (CSF) taken from patients with Alzheimer's disease compared to CSF taken from healthy control subjects (Georganopoulou et al. PNAS, 2005, 102, 2273; Pitschke et al., Nature Medicine, 1998, 4(7)).

A need exists for the use of additional biomarkers for early detection of Alzheimer's disease that will allow faster and more efficient diagnosis and evaluation of potential therapies for Alzheimer's disease. In addition, a need exists for prognosis of Alzheimer's disease as opposed to other dementias from a clinical diagnosis of Mild Cognitive Impairment (MCI). A further need exists for methods that would allow for determination of the stage of Alzheimer's disease in a patient such that treatments and care may be appropriately tailored for efficient health care regimens. In addition, the ability to evaluate the progression of Alzheimer's disease will greatly aid in the testing and evaluation of potential therapies for the disease.

### BRIEF SUMMARY OF PREFERRED EMBODIMENTS

The invention described herein meets these needs by providing methods using Aβ40 aggregates, either alone or in combination with Aβ42 monomer, Aβ40 monomer or other indicators as a biomarker for Alzheimer's disease.

Thus, in one aspect includes methods for assessing increased probability of having Alzheimer's disease in a subject under assessment including a step of obtaining a measurement of Aβ40 aggregates in a biological sample from the subject, wherein the biological sample does not include brain tissue, a fraction of brain tissue or brain homogenate. In certain embodiments of this aspect, the method further includes a step of reporting the Aβ40 aggregate measurement to a reporting means including a visual display or a printer. In other embodiments, the Aβ40 aggregate measurement includes the level of Aβ40 aggregates in the biological sample. In other embodiments, the methods further include the step of determining that a subject has an increased probability of having Alzheimer's disease if the Aβ40 aggregate level is higher than a control level threshold, wherein the control level threshold is calculated from data including the levels of Aβ40 aggregates in biological samples from a plurality of cognitively normal control subjects; wherein the subject under assessment and the control subjects are of a same species; and wherein all of the biological samples are of a same sample type. In certain embodiments of the above methods, the Alzheimer's disease is in early stage.

Another aspect includes methods for assessing increased probability of not having Alzheimer's disease in a subject under assessment including the steps of: obtaining the level of Aβ40 aggregates in a biological sample from the subject; and determining that the subject has an increased probability of not having Alzheimer's disease if the subject does not show clinical cognitive impairment and if the Aβ40 aggregate level is the same as or lower than a control level threshold; wherein the control level threshold is calculated from data including the levels of Aβ40 aggregates in biological samples from a plurality of cognitively normal control subjects; wherein the subject under assessment and the control subjects are of a same species; and wherein all of the biological samples are of a same sample type and none of the biological samples includes brain tissue, a fraction of brain tissue or brain homogenate.

A related aspect includes methods for assisting in assessing increased probability of not having Alzheimer's disease in a subject under assessment including the steps of: measuring the level of Aβ40 aggregates in a biological sample from the subject; and communicating the Aβ40 aggregate level to a different entity; wherein the different entity determines that the subject has an increased probability of not having Alzheimer's disease if the subject does not show clinical cognitive impairment and if the Aβ40 aggregate level is the same as or lower than a control level threshold; wherein the control level threshold is calculated from data including the levels of Aβ40 aggregates in biological samples from a plurality of cognitively normal control subjects; wherein the subject under assessment and the control subjects are of a same species; and wherein all of the biological samples are of a same sample type and none of the biological samples includes brain tissue, a fraction of brain tissue or brain homogenate.

Yet another aspect includes methods for monitoring disease progression in a subject with Alzheimer's disease who has not undergone any treatment for Alzheimer's disease including the steps of: obtaining the level of Aβ40 aggregates in a biological sample taken from the subject at a time after the subject is diagnosed with Alzheimer's disease; and determining that the subject has an increased probability of having progressed to an advanced stage Alzheimer's disease if (a) the Aβ40 aggregate level is lower than an early stage level of Aβ40 aggregates in the subject if available; or (b) the Aβ40 aggregate level is lower than an early stage standard if the early stage level in the subject is not available; or the subject has a decreased probability of having progressed to an advanced stage Alzheimer's disease if (a) the Aβ40 aggregate level is the same as the early stage level in the subject if available; or (b) the Aβ40 aggregate level is the same as the early stage standard if the early stage level in the subject is not available; wherein the early stage level is the level of Aβ40 aggregates in a biological sample taken from the subject at early stage of Alzheimer's disease; wherein the early stage standard is the level of Aβ40 aggregates in one or more biological samples taken from one or more standard subjects with known early stage of Alzheimer's disease; wherein the subject and the standard subjects are of a same species; and wherein all of the biological samples are of a same sample type and none of the biological samples includes brain tissue, a fraction of brain tissue or brain homogenate.

A related aspect includes methods for assisting in monitoring disease progression in a subject with Alzheimer's disease who has not undergone any treatment for Alzheimer's disease including the steps of: measuring the level of Aβ40 aggregates in a biological sample taken from the subject at a time after the subject is diagnosed with Alzheimer's disease; and communicating the Aβ40 aggregate level to a different entity; wherein the different entity determines that the subject has an increased probability of having progressed to an advanced stage Alzheimer's disease if (a) the Aβ40 aggregate level is lower than an early stage level of Aβ40 aggregates in the subject if available; or (b) the Aβ40 aggregate level is lower than an early stage standard if the early stage level in the subject is not available; or the subject has a decreased probability of having progressed to an advanced stage Alzheimer's disease if (a) the Aβ40 aggregate level is the same as the early stage level in the subject if available; or (b) the Aβ40 aggregate level is the same as the early stage standard if the early stage level in the subject is not available; wherein the early stage level is the level of Aβ40 aggregates in a biological sample taken from the subject at early stage of Alzheimer's disease; wherein the early stage standard is the level of Aβ40 aggregates in one or more biological samples taken from one or more standard subjects with known early stage of Alzheimer's disease; wherein the subject and the standard subjects are of a same species; and wherein all of the biological samples are of a same sample type and none of the biological samples includes brain tissue, a fraction of brain tissue or brain homogenate.

Still yet another aspect includes methods for assigning disease stage for a subject with Alzheimer's disease prior to any treatment for Alzheimer's disease including the steps of: obtaining the level of Aβ40 aggregates in a biological sample taken from the subject; and determining that the subject has an increased probability of having the same disease stage as that of a stage-specific standard, if available, if the Aβ40 aggregate level is close to the stage-specific standard; wherein the stage-specific standard is the level of Aβ40 aggregates in one or more biological samples taken from one or more standard subjects with a same known stage of Alzheimer's disease; wherein the subject and the standard subjects are of a same species; and wherein all of the biological samples are of a same sample type and none of the biological samples includes brain tissue, a fraction of brain tissue or brain homogenate.

A related aspect includes methods for assisting in assigning disease stage for a subject with Alzheimer's disease prior to any treatment for Alzheimer's disease including the steps of: measuring the level of Aβ40 aggregates in a biological sample taken from the subject; and communicating the Aβ40 aggregate level to a different entity; wherein the different entity determines that the subject has an increased probability of having the same disease stage as that of a stage-specific standard, if available, if the Aβ40 aggregate level is close to the stage-specific standard; wherein the stage-specific standard is the level of Aβ40 aggregates in one or more biological samples taken from one or more standard subjects with a same known stage of Alzheimer's disease; wherein the subject and the standard subjects are of a same species; and wherein all of the biological samples are of a same sample type and none of the biological samples includes brain tissue, a fraction of brain tissue or brain homogenate.

In certain embodiments of the methods relating to disease progression and assigning disease stage, the early stage standard or the stage-specific standard is the mean of levels of Aβ40 aggregates in biological samples taken from a plurality of standard subjects with a same known stage of Alzheimer's disease. In some embodiments, at least one early stage standard and one advanced stage standard are available for comparison with the Aβ40 aggregate level.

Another aspect includes methods of assessing increased probability of having Alzheimer's disease as described above and further including a step of obtaining a measurement of a second indicator of Alzheimer's disease in the subject under assessment. In certain embodiments, there is an additional step of calculating a subject index based on data including the Aβ40 aggregate measurement and the second indicator measurement. In some embodiments of the methods, after calculation of the subject index, the method further includes a step of comparing the subject index to a control index threshold, wherein the control index threshold is calculated from data including the measurements of Aβ40 aggregates in biological samples from a plurality of cognitively normal control subjects and the measurements of the second indicator in the control subjects; wherein the subject under assessment and the control subjects are of a same species; and wherein all of the biological samples are of a same sample type.

In certain embodiments, the second indicator is measured in the biological sample. In preferred embodiments, the second indicator is Aβ42 monomer or Aβ40 monomer. In certain embodiments, the second indicator is Aβ42 monomer; the subject index is a ratio of Aβ40 aggregate level to Aβ42 monomer level in the subject under assessment; and the control index threshold is a control ratio threshold which is calculated from data including the levels of Aβ40 aggregates and the levels of Aβ42 monomers in biological samples from the control subjects. In other embodiments, the second indicator is Aβ40 monomer, the subject index is a ratio of Aβ40 aggregate level to Aβ40 monomer level in the subject under assessment; and the control index threshold is a control ratio threshold which is calculated from data including the levels of Aβ40 aggregates and the levels of Aβ40 monomers in biological samples from the control subjects.

In certain embodiments of methods where the second indicator is Aβ42 monomer or Aβ40 monomer, there is an additional step of determining that the subject under assessment has an increased probability of having Alzheimer's disease if the ratio is higher than the control ratio threshold.

In certain embodiments of the methods relating to detection of a second indicator, the Alzheimer's disease is in early stage.

Another aspect includes methods for assessing increased probability of not having Alzheimer's disease in a subject under assessment including the steps of: obtaining a ratio of Aβ40 aggregate level to Aβ42 monomer level in a biological sample taken from the subject; and determining that the subject has an increased probability of not having Alzheimer's disease if the subject does not show clinical cognitive impairment and if the ratio is the same as or lower than a control ratio threshold; wherein the control ratio threshold is calculated from data including the levels of Aβ40 aggregates and the levels of Aβ42 monomers in biological samples from a plurality of cognitively normal control subjects; wherein the subject under assessment and the control subjects are of a same species; and wherein all of the biological samples are of a same sample type and none of the biological samples includes brain tissue, a fraction of brain tissue or brain homogenate.

A related aspect includes methods for assisting in assessing increased probability of not having Alzheimer's disease in a subject under assessment including the steps of: measuring the level of Aβ40 aggregates and the level of Aβ42 monomers in a biological sample taken from the subject; and communicating either the Aβ40 aggregate level and the Aβ42 monomer level or the ratio of the Aβ40 aggregate level to the Aβ42 monomer level to a different entity; wherein the different entity determines that the subject has an increased probability of not having Alzheimer's disease if the subject does not show clinical cognitive impairment and if the ratio is the same as or lower than a control ratio threshold; wherein the control ratio threshold is calculated from data including the levels of Aβ40 aggregates and the levels of Aβ42 monomers in biological samples from a plurality of cognitively normal control subjects; wherein the subject under assessment and the control subjects are of a same species; and wherein all of the biological samples are of a same sample type and none of the biological samples includes brain tissue, a fraction of brain tissue or brain homogenate.

Yet another aspect includes methods for monitoring disease progression in a subject with Alzheimer's disease who has not undergone any treatment for Alzheimer's disease including the steps of: obtaining a ratio of Aβ40 aggregate level to Aβ42 monomer level in a biological sample taken from the subject at a time after the subject is diagnosed with Alzheimer's disease; and determining that the subject has an increased probability of having progressed to an advanced stage Alzheimer's disease if (a) the ratio is lower than an early stage ratio of Aβ40 aggregate level to Aβ42 monomer level in the subject if available; or (b) the ratio is lower than an early stage ratio standard if the early stage ratio in the subject is not available; or the subject has a decreased probability of having progressed to an advanced stage Alzheimer's disease if (a) the ratio is the same as the early stage ratio in the subject if available; or (b) the ratio is the same as the early stage ratio standard if the early stage ratio in the subject is not available; wherein the early stage ratio is the ratio of Aβ40 aggregate level to Aβ42 monomer level in a biological sample taken from the subject at early stage of Alzheimer's disease; wherein the early stage ratio standard is the ratio of Aβ40 aggregate level to Aβ42 monomer level in one or more biological samples taken from one or more standard subjects with known early stage of Alzheimer's disease; wherein the subject and the standard subjects are of a same species; and wherein all of the biological samples are of a same sample type and none of the biological samples includes brain tissue, a fraction of brain tissue or brain homogenate.

A related aspect includes methods for assisting in monitoring disease progression in a subject with Alzheimer's disease who has not undergone any treatment for Alzheimer's disease including the steps of: measuring the level of Aβ40 aggregates and the level of Aβ42 monomers in a biological sample taken from the subject at a time after the subject is diagnosed with Alzheimer's disease; and communicating either the Aβ40 aggregate level and the Aβ42 monomer level or the ratio of the Aβ40 aggregate level to the Aβ42 monomer level to a different entity; wherein the different entity determines that the subject has an increased probability of having progressed to an advanced stage Alzheimer's disease if (a) the ratio of the Aβ40 aggregate level to the Aβ42 monomer level is lower than an early stage ratio of Aβ40 aggregate level to Aβ42 monomer level in the subject if available; or (b) the ratio is lower than an early stage ratio standard if the early stage ratio in the subject is not available; or the subject has a decreased probability of having progressed to an advanced stage Alzheimer's disease if (a) the ratio is the same as the early stage ratio in the subject if available; or (b) the ratio is the same as the early stage standard if the early stage ratio in the subject is not available; wherein the early stage ratio is the ratio of Aβ40 aggregate level to Aβ42 monomer level in a biological sample taken from the subject at early stage of Alzheimer's disease; wherein the early stage ratio standard is the ratio of Aβ40 aggregate level to Aβ42 monomer level in one or more biological samples taken from one or more standard subjects with known early stage of Alzheimer's disease; wherein the subject and the standard subjects are of a same species; and wherein all of the biological samples are of a same sample type and none of the biological samples includes brain tissue, a fraction of brain tissue or brain homogenate.

Still yet another aspect includes methods for assigning disease stage for a subject with Alzheimer's disease prior to any treatment for Alzheimer's disease including the steps of: obtaining a ratio of Aβ40 aggregate level to Aβ42 monomer level in a biological sample taken from the subject; and determining that the subject has an increased probability of having the same disease stage as that of a stage-specific ratio standard, if available, if the ratio is close to the ratio standard; wherein the ratio standard is the ratio of Aβ40 aggregate level to Aβ42 monomer level in one or more biological samples taken from one or more standard subjects with a same known stage of Alzheimer's disease; wherein the subject and the standard subjects are of a same species; and wherein all of the biological samples are of a same sample type and none of the biological samples includes brain tissue, a fraction of brain tissue or brain homogenate.

A related aspect includes methods for assisting in assigning disease stage for a subject with Alzheimer's disease prior to any treatment for Alzheimer's disease including the steps of: measuring the level of Aβ40 aggregates and the level of Aβ42 monomers in a biological sample taken from the subject; and communicating either the Aβ40 aggregate level and the Aβ42 monomer level or the ratio of the Aβ40 aggregate level to the Aβ42 monomer level to a different entity; wherein the different entity determines that the subject has an increased probability of having the same disease stage as that of a stage-specific ratio standard, if available, if the ratio is close to the ratio standard; wherein the ratio standard is the ratio of Aβ40 aggregate level to Aβ42 monomer level in one or more biological samples taken from one or more standard subjects with a same known stage of Alzheimer's disease; wherein the subject and the standard subjects are of a same species; and wherein all of the biological samples are of a same sample type and none of the biological samples includes brain tissue, a fraction of brain tissue or brain homogenate.

In certain embodiments of the methods relating to Aβ40 aggregate ratios used as a biomarker for disease progression or stage, early stage ratio standard or the stage-specific ratio standard is the mean of the ratios of Aβ40 aggregate level to Aβ42 monomer level in biological samples taken from a plurality of standard subjects with a same known stage of Alzheimer's disease. In certain embodiments, at least one early stage ratio standard and one advanced stage ratio standard are available for comparison with the ratio.

In yet another aspect, the methods described herein include methods for assessing increased probability of MCI progressing to Alzheimer's disease including a step of obtaining a measurement of Aβ40 aggregates in a biological sample from the subject, wherein the biological sample does not include brain tissue, a fraction of brain tissue or brain homogenate. In certain embodiments of this aspect, the method further includes a step of reporting the Aβ40 aggregate measurement to a reporting means including a visual display or a printer. In other embodiments, the Aβ40 aggregate measurement includes the level of Aβ40 aggregates in the biological sample. In other embodiments, the methods further include the step of determining that a subject has an increased probability of MCI progressing to Alzheimer's disease if the Aβ40 aggregate level is higher than a control level threshold, wherein the control level threshold is calculated from data including the levels of Aβ40 aggregates in biological samples from a plurality of cognitively normal control subjects; wherein the subject under assessment and the control subjects are of a same species; and wherein all of the biological samples are of a same sample type. In certain embodiments of the above methods, the Alzheimer's disease is in early stage.

Another aspect includes methods of assessing increased probability of MCI progressing to Alzheimer's disease as described above and further including a step of obtaining a measurement of a second indicator of MCI progressing to Alzheimer's disease in the subject under assessment. In certain embodiments, there is an additional step of calculating a subject index based on data including the Aβ40 aggregate measurement and the second indicator measurement. In some embodiments of the methods, after calculation of the subject index, the method further includes a step of comparing the subject index to a control index threshold, wherein the control index threshold is calculated from data including the measurements of Aβ40 aggregates in biological samples from a plurality of cognitively normal control subjects and the measurements of the second indicator in the control subjects; wherein the subject under assessment and the control subjects are of a same species; and wherein all of the biological samples are of a same sample type.

In certain embodiments, the second indicator is measured in the biological sample. In preferred embodiments, the second indicator is Aβ42 monomer. In certain embodiments, the second indicator is Aβ42 monomer; the subject index is a ratio of Aβ40 aggregate level to Aβ42 monomer level in the subject under assessment; and the control index threshold is a control ratio threshold which is calculated from data including the levels of Aβ40 aggregates and the levels of Aβ42 monomers in biological samples from the control subjects. In certain embodiments of methods where the second indicator is Aβ42 monomer, there is an additional step of determining that the subject under assessment has an increased probability of MCI progressing to Alzheimer's disease if the ratio is higher than the control ratio threshold. In certain embodiments of the methods relating to detection of a second indicator, the Alzheimer's disease is in early stage.

Another aspect includes methods for assessing increased probability of MCI progressing to Alzheimer's disease in a subject under assessment including the step of obtaining a measurement of Aβ40 aggregates in a biological sample from a subject, determining that the subject has an increased probability of MCI progressing to Alzheimer's disease if the Aβ40 aggregate level is higher than a control level threshold, wherein the control level threshold is calculated from data including the levels of Aβ40 aggregates in biological samples from a plurality of cognitively normal control subjects; wherein the subject under assessment and the control subjects are of a same species; and wherein all of said biological samples include CSF, and wherein none of the biological samples include brain tissue, a fraction of brain tissue or brain homogenate.

A related aspect includes methods for assisting in assessing increased probability of MCI progressing to Alzheimer's disease in a subject under assessment including the steps of: measuring the level of Aβ40 aggregates in a biological sample from the subject; and communicating the Aβ40 aggregate level to a different entity; wherein the different entity determines that the subject has an increased probability of MCI progressing to Alzheimer's disease if the Aβ40 aggregate level is higher than a control level threshold; wherein the control level threshold is calculated from data including the levels of Aβ40 aggregates in biological samples from a plurality of cognitively normal control subjects; wherein the subject under assessment and the control subjects are of a same species; and wherein all of the biological samples include CSF and none of the biological samples include brain tissue, a fraction of brain tissue or brain homogenate.

Yet another aspect includes methods for assessing increased probability of MCI progressing to Alzheimer's disease in a subject under assessment including the steps of: obtaining a ratio of Aβ40 aggregate level to Aβ42 monomer level in a biological sample taken from the subject; and determining that the subject has an increased probability of MCI progressing to Alzheimer's disease if the ratio is higher than a control ratio threshold; wherein the control ratio threshold is calculated from data comprising the levels of Aβ40 aggregates and the levels of Aβ42 monomers in biological samples from a plurality of cognitively normal control subjects; wherein the subject under assessment and the control subjects are of a same species; and wherein all of the biological samples include CSF and none of the biological samples comprises brain tissue, a fraction of brain tissue or brain homogenate.

A related aspect includes methods for assisting in assessing increased probability of MCI progressing to Alzheimer's disease in a subject under assessment including the steps of: determining a ratio of Aβ40 aggregate level to Aβ42 monomer level in a biological sample from the subject; and communicating either the Aβ40 aggregate level and the Aβ42 monomer level or the ratio of the Aβ40 aggregate level to the Aβ42 monomer level to a different entity; wherein the different entity determines that the subject has an increased probability of MCI progressing to Alzheimer's disease if the ratio is higher than a control ratio threshold; wherein the control ratio threshold is calculated from data including the levels of Aβ40 aggregates and the levels of Aβ42 monomers in biological samples from a plurality of cognitively normal control subjects; wherein the subject under assessment and the control subjects are of a same species; and wherein all of the biological samples include CSF and none of the biological samples include brain tissue, a fraction of brain tissue or brain homogenate.

The methods described above can be practiced using various different subjects. In certain embodiments of any of the above described methods, the subject under assessment or the subject with Alzheimer's disease is a human. In other embodiments of the above methods the subject under assessment or the subject with Alzheimer's disease is a non-human animal. In certain embodiments, the subject under assessment or the subject with Alzheimer's disease is alive or the control subjects or the standard subjects are alive.

The methods described above can be practiced on various different biological samples. In certain embodiments, the biological sample from the subject under assessment or the subject with Alzheimer's disease contains bodily fluid or bodily tissue. In specific embodiments, the biological sample can contain whole blood, blood fractions, blood components, plasma, platelets, serum, cerebrospinal fluid (CSF), bone marrow, urine, tears, milk, lymph fluid, organ tissue, nervous system tissue, non-nervous system tissue, muscle tissue, biopsy, necropsy, fat biopsy, fat tissue, cells, feces, placenta, spleen tissue, lymph tissue, pancreatic tissue, bronchoalveolar lavage (BAL), or synovial fluid. In preferred embodiments, the biological sample can contain plasma, serum, CSF, or urine.

In embodiments of the methods where the sample contains bodily fluid or tissues, the Aβ40 aggregates are preferably circulating. In certain embodiments, the biological sample is collected using a same method in the same manner as the biological samples from the control subjects or standard subjects.

In certain embodiments of any of the methods described above, the biological sample contains bodily fluid; and the Aβ40 aggregate measurement or the Aβ40 aggregate level is obtained by a method including the steps of: contacting the bodily fluid with an aggregate-specific binding reagent under conditions that allow binding of the reagent to Aβ40 aggregates, if present, to form a complex; and detecting Aβ40 aggregates, if any, in the subject biological sample by its binding to the reagent; wherein the reagent is attached to a solid support and binds preferentially to aggregate over monomer when attached to the solid support.

In other embodiments, the biological sample contains bodily tissue; and the Aβ40 aggregate measurement or the Aβ40 aggregate level is obtained by a method including the steps of: providing a homogenate of the bodily tissue; contacting the homogenate with an aggregate-specific binding reagent under conditions that allow binding of the reagent to Aβ40 aggregates, if present, to form a complex; and detecting Aβ40 aggregates, if any, in the subject biological sample by its binding to the reagent; wherein the reagent is attached to a solid support and binds preferentially to aggregate over monomer when attached to the solid support.

In certain embodiments, the detecting step includes the substeps of: separating the complex formed by the reagent and Aβ40 aggregates from unbound monomers of Aβ40, if present; optionally, dissociating Aβ40 aggregates from the complex; and detecting Aβ40 aggregates. In other embodiments, the detecting step includes the substeps of: separating the complex formed by the reagent and Aβ40 aggregates from unbound monomers of Aβ40, if present, and removing the unbound monomers of Aβ40; denaturing the Aβ40 aggregates present in the complex to form Aβ40 monomers; and detecting Aβ40 monomers. The Aβ40 aggregates may be detected by a detection reagent after the complex is separated from unbound monomers, if present. The detection reagent may be detectably labeled.

In still other embodiments, the Aβ40 aggregate measurement or the Aβ40 aggregate level is obtained by a method that employs seeded multimerization. In embodiments of methods using an aggregate-specific binding reagent, the reagent may be a peptide, peptoid or dendron; the solid support may be nitrocellulose, polystyrene latex, polyvinyl fluoride, diazotized paper, nylon membrane, activated bead, magnetically responsive bead, titanium oxide, silicon oxide, polysaccharide bead, polysaccharide membrane, agarose, glass, polyacrylic acid, polyethyleneglycol, polyethyleneglycol-polystyrene hybrid, controlled pore glass, glass slide, gold bead, or cellulose; and the reagent may be detectably labeled

### BRIEF DESCRIPTION OF THE DRAWINGS

FIG. 1 shows the steps of the Misfolded Protein Assay (MPA) used to detect Aβ aggregates.

FIG. 2 shows the Aβ40 aggregate results obtained with 8 AD and 8 control CSF samples coming from the commercial source Analytical Biological Sciences (ABS). Panel A: There is a statistically significant difference in Aβ40 aggregate results between control and AD groups; Panel B: Based on clinical cognitive mini mental state examination (MMSE) test scores, AD groups were separated according to disease stages. ANOVA shows that these samples do not come from a single population. Individual t-tests which demonstrate significant difference between groups are shown. Y-axis in all graphs is the relative light units detected in the MPA.

FIG. 3 shows the Aβ40 aggregate results obtained with 35 AD and 23 control CSF samples coming from a commercial source (ABS). Panel A: There is a statistically significant difference in Aβ40 aggregate results between control and AD groups; Panel B: AD samples were separated into disease stages by the vendor. ANOVA shows that these samples do not come from a single population. Individual t-tests which demonstrate significant difference between groups are shown. Y-axis in all graphs is the relative light units detected in the MPA.

FIG. 4 shows the Aβ40 aggregate results obtained with 26 AD and 10 matched-control CSF samples obtained from a university research hospital. Panel A: There is a statistically significant difference in Aβ40 aggregate results between control and AD groups. Y-axis is the relative light units detected in the MPA. Panel B: Based on clinical cognitive mini mental state examination (MMSE) test scores, AD groups were separated according to disease stages. ANOVA shows that these samples do not come from a single population. Individual t-tests which demonstrate significant difference between groups are shown.

FIG. 5 shows the results for Aβ42 monomer and Aβ40 aggregate/Aβ42 monomer ratio obtained with the same 26 AD and 10 matched-control CSF samples shown in FIG. 4. Panel A: Statistically significant decrease in Aβ42 monomer signal in AD samples is measured in pre-capture CSF. Y-axis is the pg/ml of Aβ42 monomer measured by immunoassay in pre-capture CSF. Panel B: A statistically significant increase in Aβ40 aggregate/Aβ42 monomer ratio in the AD samples is observed. Panel C: Based on clinical cognitive mini mental state examination (MMSE) test scores, AD groups were separated according to disease stages. ANOVA shows that these samples do not come from a single population. Individual t-tests which demonstrate significant difference between groups are shown. Y-axis in Panels B and C is the ratio of relative light units of Aβ40 signal detected in the MPA to pg/ml of Aβ42 monomer measured by immunoassay in pre-capture CSF.

FIG. 6 shows ROC curves for AD vs. control data from the same clinical sample set shown in FIG. 4 (26 AD and 10 matched-control CSF samples).

FIG. 7 shows ROC curves for early stage AD vs. control data from the same clinical sample set shown in FIG. 4 (13 early AD and 10 matched-control CSF samples).

FIG. 8 shows results for Aβ40 monomer and Aβ40 aggregate/Aβ40 monomer ratio obtained with the same 26 AD and 10 matched-control CSF samples shown in FIG. 4. Panel A: No statistically significant decrease in Aβ40 monomer signal was observed in AD measured in pre-capture CSF. Y-axis in graphs in Panels A is pg/ml of Aβ40 monomer measured by immunoassay in pre-capture CSF. Panel B: A statistically significant increase in Aβ40 aggregate level/Aβ40 monomer level ratio in the AD samples is observed. Y-axis is the ratio of relative light units of Aβ40 signal detected in the MPA to pg/ml of Aβ40 monomer measured by immunoassay in pre-capture CSF.

FIG. 9 shows the Aβ40 aggregate results obtained with a larger set of clinical samples from the same university research hospital (47 AD, 71 MCI, and 21 control). Panel A: Samples were initially grouped into 3 different populations based on clinical diagnosis at the time of CSF collection. While there is no statistical difference among the 3 groups by ANOVA, there is a statistically significant difference in Aβ40 aggregate results between control and AD groups. Panel B: MCI samples were further subdivided based on follow up clinical diagnosis made after the CSF sample collection. There is a statistically significant difference in Aβ40 aggregate results between control and AD groups. Panel C: Subdivided MCI samples after removal of the single high value outlier in the MCI to AD group. ANOVA shows that these samples do not come from a single population. Individual t-tests which demonstrate significant difference between groups are shown. Y-axis in all graphs is the relative light units detected in the MPA.

FIG. 10 shows the results for Aβ42 monomer and Aβ40 aggregate/Aβ42 monomer ratio obtained with the same 47 AD, 71 MCI, and 21 matched-control CSF samples shown in FIG. 9. Panel A: There are statistically significant decreases in Aβ42 monomer signal measured in pre-capture CSF in both the MCI and AD samples. Panel B: There is a statistically significant differences in Aβ40 aggregate/Aβ42 monomer ratio results between control and both MCI and AD groups. Panel C: There is a statistically significant difference in Aβ42 monomer results between control and both MCI which later progressed to AD and AD groups. Panel D: There is a statistically significant difference in Aβ40 aggregate/Aβ42 monomer results between control and both MCI which later progresses to AD and AD groups. In all panels ANOVA shows that these samples do not come from a single population. Individual t-tests which demonstrate significant difference between groups are shown. Y-axis in graphs in Panels A and C is pg/ml of Aβ42 monomer measured by immunoassay in pre-capture CSF. Y-axis in Panels B and D is the ratio of relative light units of Aβ40 signal detected in the MPA to pg/ml of Aβ42 monomer measured by immunoassay in pre-capture CSF.

FIG. 11 shows ROC curves for the control vs. AD data from the same larger clinical sample set shown in FIG. 9 (47 AD, 71 MCI, and 21 control).

FIG. 12 shows ROC curves for the control vs. MCI which progresses to AD and AD data from the same larger clinical sample set shown in FIG. 9 (47 AD, 71 MCI, and 21 control).

FIG. 13 shows the Aβ40 aggregate and Aβ42 monomer results obtained with another, different set of CSF samples obtained from a university research hospital. Panel A: There is no statistically significant difference in Aβ40 aggregate results between control and AD groups. Y-axis is the relative light units detected in the MPA. Panel B: There is no statistically significant difference in Aβ42 monomer results between control and AD groups. Y-axis is the pg/ml of Aβ42 monomer measured by immunoassay in pre-capture CSF. Panel C: There is no statistically significant difference in Aβ40 aggregate/Aβ42 monomer results between control and AD groups. Y-axis is the relative light units of Aβ40 signal detected in the MPA to pg/ml of Aβ42 monomer measured by immunoassay in pre-capture CSF.

FIG. 14 depicts the amount of Aβ40 aggregates detected by the Misfolded Protein Assay in the supernatant and pellet of Alzheimer's Disease CSF and normal CSF centrifuged at 16,000 g for 10 minutes or 134,000 g for 1 hour. Legend: Small checks: total amount Aβ40; Large checks: 16,000 g supernatant; Horizontal line: 16,000 pellet; Vertical line: 134,000 g supernatant, Diagonal line: 134,000 g pellet.

### DETAILED DESCRIPTION

This invention relates in part to the discovery of biomarkers which can be used in assessing the probability of Alzheimer's disease, in monitoring the progression of Alzheimer's disease, in assigning Alzheimer's disease stage, and in early diagnosis of Alzheimer's disease, in particular assessing the probability of MCI patients progressing to Alzheimer's disease. In particular, the invention includes methods involving the use of aggregates of Aβ40 alone or in combination with monomers of Aβ40 or Aβ42 in assessing the probability of Alzheimer's disease, in monitoring the progression of Alzheimer's disease, and in assigning Alzheimer's disease stage.

The practice of the present invention will employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Handbook of Surface and Colloidal Chemistry (Birdi, K. S. ed., CRC Press, 1997); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Peters and Dalrymple, Fields Virology (2d ed), Fields et al. (eds.), B.N. Raven Press, New York, N.Y.

It is understood that the reagents and methods of this invention are not limited to particular formulations or process parameters as such may, of course, vary. It is also to be understood that the terminology used herein is for the purpose of describing particular embodiments of the invention only, and is not intended to be limiting.

### I. Definitions

In order to facilitate an understanding of the invention, selected terms used in the application will be discussed below.

“Biomarker” as used herein encompasses, without limitation, proteins, nucleic acids, and metabolites, together with their polymorphisms, mutations, variants, modifications, subunits, fragments, protein-ligand complexes, and degradation products, protein-ligand complexes, elements, related metabolites, and other analytes or sample-derived measures that are associated with a biological state. Biomarkers can also include mutated proteins or mutated nucleic acids. The term “analyte” as used herein can mean any substance to be measured and can encompass electrolytes and elements, such as calcium.

As used herein, “Aβ40 monomer” refers to total undenatured Aβ40 protein that is detected by an antibody that is specific to an epitope on Aβ40 that is not exposed when the protein is aggregated. “Aβ42 monomer” refers to total undenatured Aβ42 protein that is detected by an antibody that is specific to an epitope on Aβ42 that is not exposed when the protein is aggregated.

As used herein, the term “aggregate” refers to a complex containing more than one copy of a non-native conformer of a protein that arises from non-native interactions among the conformers. Aggregates may contain multiple copies of the same protein, multiple copies of more than one protein, and additional components including, without limitation, glycoproteins, lipoproteins, lipids, glycans, nucleic acids, and salts. Aggregates may exist in structures such as inclusion bodies, plaques, or aggresomes. Aggregates may be on or off pathway with repect to fibril formation. Some examples of aggregates are amorphous aggregates, oligomers, and fibrils. Amorphous aggregates are typically disordered and insoluble. An “oligomer” as used herein contains more than one copy of a non-native conformer of a protein. Typically, they contain at least 2 monomers, but no more than 1000 monomers, or in some cases, no more than 10⁶ monomers. Oligomers include small micellar aggregates and protofibrils. Small micellar aggregates are typically soluble, ordered, and spherical in structure. Protofibrils are also typically soluble, ordered aggregates with beta-sheet structure. Protofibrils are typically curvilinear in structure and contain at least 10, or in some cases, at least 20 monomers. Fibrils are typically insoluble and highly ordered aggregates. Fibrils typically contain hundreds to thousands of monomers. Fibrils include, for example, amyloids, which exhibit cross-beta sheet structure and can be identified by apple-green birefringence when stained with Congo Red and seen under polarized light. When contained in a single sample, aggregates such as amorphous aggregates, oligomers, and fibrils may be separated by centrifugation. For example, centrifugation at 14,000×g for 10 minutes will typically remove only very large aggregates, such as large fibrils and amorphous aggregates (10-1000 MDa), and centrifugation at 100,000×g for one hour will typically remove aggregates larger than 1 MDa, such as smaller fibrils and amorphous aggregates. Size and solubility of aggregates will affect the sedimentation velocity required for separation. In preferred embodiments of the invention, aggregates contain Aβ40.

The term “aggregate-specific binding reagent” or “ASB reagent” refers to any type of reagent, including but not limited to peptides, peptoids, and dendrons, which binds preferentially to an aggregate compared to monomer when attached to a solid support at certain charge densities. The binding may be due to increased affinity, avidity, or specificity. For example, in certain embodiments, the aggregate-specific binding reagents described herein bind preferentially to aggregates but, nonetheless, may also be capable of binding monomers at a weak, yet detectable, level. Typically, weak binding, or background binding, is readily discernible from the preferential interaction with the aggregate of interest, e.g., by use of appropriate controls. In general, aggregate-specific binding reagents used in methods of the invention bind aggregates in the presence of an excess of monomers. Preferably, ASB reagents bind aggregates with an affinity/avidity that is at least about two times higher than the binding affinity/avidity for monomer.

An aggregate-specific binding reagent is said to “bind” with another peptide or protein if it binds specifically, non-specifically or in some combination of specific and non-specific binding. A reagent is said to “bind preferentially” to an aggregate if it binds with greater affinity, avidity, and/or greater specificity to the aggregate than to monomer. The terms “bind preferentially,” “preferentially bind,” “bind selectively,” “selectively bind,” and “selectively capture” are use interchangeably herein.

The term “Alzheimer's disease (AD) protein” or “AD protein” are used interchangeably herein to refer to both the aggregate (variously referred to as pathogenic protein form, pathogenic isoform, pathogenic Alzheimer's disease protein, and Alzheimer's disease conformer) and the non-aggregate (variously referred to as monomer, normal cellular form, non-pathogenic isoform, non-pathogenic Alzheimer's disease protein), as well as the denatured form and various recombinant forms of the Alzheimer's disease protein which may not have either the pathogenic conformation or the normal cellular conformation. Exemplary Alzheimer's disease proteins include Aβ and the tau protein.

The terms “amyloid-beta,” “amyloid-β,” “Abeta”, “Aβ,” “Aβ42”, “Aβ40,” “Aβx-42,” “Aβx-40,” and “Aβ40/42” as used herein all refer to amyloid-β peptides, which are a family of up to 43 amino acids in length found extracellularly after the cleavage of the amyloid precursor protein (APP). The term Aβ is used to refer generally to the amyloid-β peptides in any form. The term “Aβ40” refers to “Aβx-40.” The term “Aβ42” refers to “Aβx-42.” Preferably, x is from 1 to 17. More preferably, x equals 1, 2, 3, 4, 5, 6, 7, 8, 9, 10, 11, 12, 13, 14, 15, 16, or 17. The term “Aβ1-42” refers to a fragment corresponding to amino acids 1 to 42 of Aβ (amino acids 597-638 of the APP sequence (Johnson-Wood et al (1997) PNAS 94, 1550-1555)). The term “Aβ1-40” refers to a fragment corresponding to amino acids 1 to 40 of Aβ (amino acids 597-636 of the APP sequence). The term Aβ40/42 is used to refer to both the Aβ40 and Aβ42 isoforms.

“Indicator” as used herein refers to any factor that is associated with a biological state either alone or in combination with other indicators. Indicators include biomarkers as well as non-biological sample derived factors associated with a biological state, such as non-analyte physiological markers of health status, or other factors or markers not measured from biological samples, such as “clinical parameters” defined herein. Indicators also include any calculated indices created mathematically or combinations of any one or more of the foregoing measurements, including temporal trends and differences.

“Clinical parameters” as used herein include all non-sample or non-analyte biomarkers of subject health status or other characteristics, such as, without limitation, age, gender, family medical history, previous diagnosis of MCI, ApoE genotype, education level, and lifestyle factors such as high blood pressure, high cholesterol, and poorly controlled diabetes. Clinical parameters for Alzheimer's disease also include outcomes from clinical tests known to those of skill in the art, including the Alzheimer's Disease Assessment Scale-Cognitive (ADAS-Cog), Clinical Dementia Rating (CDR, including global and sum-of-boxes CDRs), Memory Box score, and the Mini-Mental State Examination (MMSE).

“TN” is true negative, which for a disease assessment test means classifying a non-disease or normal control correctly.

“TP” is true positive, which for a disease assessment test means correctly classifying a disease subject.

“FN” is false negative, which for a disease assessment test means classifying a disease subject incorrectly as non-disease or normal.

“FP” is false positive, which for a disease assessment test means classifying a non-disease or normal control incorrectly as having disease.

“Accuracy” refers to the degree of conformity of a measured or calculated quantity (a test reported value) to its actual (or true) value. Clinical accuracy relates to the proportion of true outcomes (true positives (TP) or true negatives (TN) versus misclassified outcomes (false positives (FP) or false negatives (FN)), and may be stated as a sensitivity, specificity, positive predictive values (PPV) or negative predictive values (NPV), or as a likelihood, odds ratio, among other measures. “Test accuracy” as used herein is calculated by (TP+TN)/(TP+TN+FP+FN).

“Sensitivity” is calculated by TP/(TP+FN) or the true positive fraction of disease subjects.

“Specificity” is calculated by TN/(TN+FP) or the true negative fraction of non-disease or normal controls.

“Negative predictive value” or “NPV” is calculated by TN/(TN+FN) or the true negative fraction of all negative test results. It also is inherently impacted by the prevalence of the disease and pre-test probability of the population intended to be tested. See, e.g., O'Marcaigh A S, Jacobson R M, “Estimating The Predictive Value Of A Diagnostic Test, How To Prevent Misleading Or Confusing Results,” Clin. Ped. 1993, 32(8): 485-491, which discusses specificity, sensitivity, and positive and negative predictive values of a test, e.g., a clinical diagnostic test. Often, for binary disease state classification approaches using a continuous diagnostic test measurement, the sensitivity and specificity is summarized by Receiver Operating Characteristics (ROC) curves according to Pepe et al, “Limitations of the Odds Ratio in Gauging the Performance of a Diagnostic, Prognostic, or Screening Marker,” Am. J. Epidemiol 2004, 159 (9): 882-890, and summarized by the Area Under the Curve (AUC) or c-statistic, an indicator that allows representation of the sensitivity and specificity of a test, assay, or method over the entire range of test (or assay) cut points with just a single value. See also, e.g., Shultz, “Clinical Interpretation Of Laboratory Procedures,” chapter 14 in Teitz, Fundamentals of Clinical Chemistry, Burtis and Ashwood (eds.), 4th edition 1996, W.B. Saunders Company, pages 192-199; and Zweig et al., “ROC Curve Analysis: An Example Showing The Relationships Among Serum Lipid And Apolipoprotein Concentrations In Identifying Subjects With Coronory Artery Disease,” Clin. Chem., 1992, 38(8): 1425-1428. An alternative approach using likelihood functions, odds ratios, information theory, predictive values, calibration (including goodness-of-fit), and reclassification measurements is summarized according to Cook, “Use and Misuse of the Receiver Operating Characteristic Curve in Risk Prediction,” Circulation 2007, 115: 928-935. Hazard ratios and absolute and relative risk ratios within subject cohorts defined by a test are a further measurement of clinical accuracy and utility. In this last, multiple methods are frequently used to defining abnormal or disease values, including reference limits, discrimination limits, and risk thresholds as per Vasan, “Biomarkers of Cardiovascular Disease Molecular Basis and Practical Considerations,” Circulation 2006, 113: 2335-2362.

“Positive predictive value” or “PPV” is calculated by TP/(TP+FP) or the true positive fraction of all positive test results. It is inherently impacted by the prevalence of the disease and pre-test probability of the population intended to be tested.

The terms “formula,” “algorithm,” and “model” are used interchangeably for any mathematical equation, algorithmic, analytical or programmed process, or statistical technique that takes one or more continuous or categorical inputs and calculates an output value, sometimes referred to as an “index” or “index value.” Non-limiting examples of “formulas” include sums, ratios, and regression operators, such as coefficients or exponents, biomarker value transformations and normalizations (including, without limitation, those normalization schemes based on clinical parameters, such as gender, age, or ethnicity), rules and guidelines, statistical classification models, and neural networks trained on historical populations. Of particular use for the biomarkers are linear and non-linear equations and statistical classification analyses to determine the relationship between levels of biomarkers detected in a subject sample and the subject's probability of having Alzheimer's disease. For diagnostic panel and combination construction, of particular interest are structural and synactic statistical classification algorithms, and methods of risk index construction, utilizing pattern recognition features, including established techniques such as cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shruken Centroids (SC), StepAIC, Kth-Nearest Neighbor, Boosting, Decision Trees, Neural Networks, Bayesian Networks, Support Vector Machines, and Hidden Markov Models, Linear Regression or classification algorithms, Nonlinear Regression or classification algorithms, analysis of variants (ANOVA), hierarchical analysis or clustering algorithms; hierarchical algorithms using decision trees; kernel based machine algorithms such as kernel partial least squares algorithms, kernel matching pursuit algorithms, kernel Fisher's discriminate analysis algorithms, or kernel principal components analysis algorithms, among others. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other studies, or cross-validated in the study they were originally trained in, using such techniques as Leave-One-Out (LOO) and 10-Fold cross-validation (10-Fold CV).

As used herein “same as” means that there is meant that there is no statistically significant difference between two measurements.

By “statistically significant,” it is meant that the alteration is greater than what might be expected to happen by chance alone (which could be a “false positive”). Statistical significance can be determined by any method known in the art. Commonly used measures of significance include the p-value, which presents the probability of obtaining a result at least as extreme as a given data point, assuming the data point was the result of chance alone. A result is often considered highly significant at a p-value of 0.05 or less.

A “subject” in the context of the present invention is preferably a mammal. The mammal can be a human, non-human primate, mouse, rat, dog, cat, horse, or cow, but are not limited to these examples. Mammals other than humans can be advantageously used as subjects that represent animal models of Alzheimer's disease. A subject can be male or female. A subject can be one who has been previously diagnosed or identified as having Alzheimer's disease. Preferably, a subject has not already undergone, or is not undergoing, a therapeutic intervention for Alzheimer's disease. Alternatively, a subject can also be one who has not been previously diagnosed as having Alzheimer's disease. For example, a subject can be one who exhibits one or more risk factors for Alzheimer's disease, or a subject who does not exhibit Alzheimer's disease risk factors, or a subject who is asymptomatic for Alzheimer's disease. A subject can also be one who is suffering from or at risk of developing Alzheimer's disease.

“Peptoid” is used generally to refer to a peptide mimic that contains at least one, preferably two or more, amino acid substitutes, preferably N-substituted glycines. Peptoids are described in, inter alia, U.S. Pat. No. 5,811,387. As used herein, a “peptoid reagent” is a molecule having an amino-terminal region, a carboxy-terminal region, and at least one “peptoid region” between the amino-terminal region and the carboxy-terminal region. The amino-terminal region refers to a region on the amino-terminal side of the reagent that typically does not contain any N-substituted glycines. The amino-terminal region can be H, alkyl, substituted alkyl, acyl, an amino protecting group, an amino acid, a peptide, or the like. The carboxy-terminal region refers to a region on the carboxy-terminal end of the peptoid that does not contain any N-substituted glycines. The carboxy-terminal region can include H, alkyl, alkoxy, amino, alkylamino, dialkylamino, a carboxy protecting group, an amino acid, a peptide, or the like.

“Dendron” as used herein is a branched polymer with little structural similarity to peptides.

“Seeded multimerization” as used herein refers to the progressive deposition and transformation of soluble Aβ monomers into aggregates. Seeded multimerization of monomers is characterized by slow-nucleation and fast-growth kinetics.

“Level” as used herein refers to a relative level.

“Bodily fluids” as used herein include circulating and non-circulating fluids. Examples of circulating fluids include blood, CSF, and lymph fluid. Examples of non-circulating fluids include synovial fluid.

“MCI” as used herein refers to Mild Cognitive Impairment. MCI is a clinical diagnosis of cognitive impairment advocated by Petersen [Peteresemn R C (2004) Mild cognitive impairment as a diagnostic entity. J Intern Med 256, 183-194] and includes subjective memory complaint, objective memory impairment confirmed by a physician, preservation of general cognitive functioning with a MMSE score of ≧24, minimal impairment of daily life activities, and failure to meet the DSM-IIIR criteria of dementia. Patients with early stage of AD who are clinically diagnosed as MCI and the subset of MCI patients who progress to AD later are likely the similar group of patients, but are sometimes classified differently by different clinicians.

### II. Methods of the Invention

### Methods for Assessing Increased Probability of Having Alzheimer's Disease

The invention described herein provides methods for assessing increased probability of Alzheimer's disease in a subject under assessment. These methods are based on the discovery that levels of Aβ40 aggregates in samples which do not include brain tissue, a fraction of brain tissue, or brain homogenate from a subject are correlated with the probability of the subject having Alzheimer's disease. Assessing increased probability of Alzheimer's disease includes determining the probability or chance that a subject has the disease or predicting whether or not a subject will develop the disease. Assessing increased probability of Alzheimer's disease further includes, for example, diagnosing the disease or providing a prognosis for the disease.

The invention described herein provides methods for assessing increased probability of Alzheimer's disease in a subject under assessment by obtaining a measurement of Aβ40 aggregates in a biological sample from the subject, wherein the biological sample does not include brain tissue, a fraction of brain tissue, or brain homogenate.

Methods for assessing increased probability of Alzheimer's disease in a subject under assessment may include the step of reporting the Aβ40 aggregate measurement to a reporting means containing a visual display or a printer. Reporting the Aβ40 aggregate measurement may occur manually or automatically. For example, the Aβ40 aggregate measurement may be manually inputed into a reporting means, or the Aβ40 aggregate measurement may be obtained in a way that allows for subsequent automatic reporting of the measurement to a reporting means. Suitable reporting means include, for example, a conventional computer processor, a computer processor directly linked to a measuring means, or a computer processor remotely linked to a measuring means such as through a wireless network.

In certain embodiments, the Aβ40 aggregate measurement includes the level of Aβ40 aggregates in the biological sample. Methods for assessing increased probability of Alzheimer's disease in a subject under assessment may include the step of determining that the subject has an increased probability of Alzheimer's disease if the Aβ40 aggregate level is higher than a control level threshold. The threshold is calculated from data including the levels of Aβ40 aggregates in biological samples from a plurality of cognitively normal control subjects. The subject under assessment and the control subjects are of the same species, and all of the biological samples are of a same sample type

Control subjects to be used in methods of the invention are cognitively normal. The determination that a subject is cognitively normal may be made according to methods known to one of skill in the art. The control subjects as used herein show no sign of impairment in commonly used clinical tests for cognitive function known to a person of skill in the art. Some examples of such clinical tests are MMSE, CDR, Memory Box score, and ADAS-Cog. Typically, control subjects will also be chosen based on other factors such as age and gender. For example, if possible, control subjects will be chosen which match the age and gender of the subject under assessment.

Threshold Calculation

The threshold may be calculated by any models known by one of skill in the art to be useful for manipulating data to create a meaningful numerical summary of the data. For example, the threshold may be calculated simply by averaging the level of Aβ40 aggregates in biological samples from a plurality of cognitively normal control subjects.

In some cases, the threshold will be used for a diagnostic test. In such cases, the threshold may be calculated from a model that incorporates preferred diagnostic performance parameters, such as accuracy, sensitivity, specificity, positive predictive value, and negative predictive value. In diagnosis of disease, changing the threshold value of a test or assay usually changes the sensitivity and specificity in a qualitatively inverse relationship. Therefore, in assessing the accuracy and usefulness of a proposed medical test, assay, or method for assessing a subject's condition, one should always take both sensitivity and specificity into account and be mindful of what the threshold is at which the sensitivity and specificity are being reported because sensitivity and specificity may vary significantly over the range of thresholds. A preferred sensitivity value for use in determining a control level threshold is at least 65%, desirably at least 70%, more desirably at least 75%, preferably at least 80%, more preferably at least 85%, and most preferably at least 90%. A preferred specificity value for use in determining a control level threshold is at least 65%, desirably at least 70%, more desirably at least 75%, preferably at least 80%, more preferably at least 85%, and most preferably at least 90%.

Use of statistics such as AUC (area under the ROC curve for the test), encompassing all potential threshold values, is preferred for most categorical risk measures using the invention, while for continuous risk measures, statistics of goodness-of-fit and calibration to observed results or other gold standards, are preferred.

An acceptable degree of diagnostic accuracy for assessing probability of Alzheimer's disease is one in which the AUC is at least 0.60, desirably at least 0.65, more desirably at least 0.70, preferably at least 0.75, more preferably at least 0.80, and most preferably at least 0.85.

A very high degree of diagnostic accuracy for assessing probability of Alzheimer's disease is one in which the AUC (area under the ROC curve for the test) is at least 0.80, desirably at least 0.85, more desirably at least 0.875, preferably at least 0.90, more preferably at least 0.925, more preferably at least 0.95, more preferably at least 0.98, and most preferably at least 0.99.

The predictive value of any assessment or test depends both on the sensitivity and specificity of the test, and on the prevalence of the condition in the population being tested. This notion, based on Bayes' theorem, provides that the greater the likelihood that the condition being screened for is present in a subject or in the population (pre-test probability), the greater the validity of a positive test and the greater the likelihood that the result is a true positive. Thus, the problem with using any test in any population where there is a low likelihood of the condition being present is that a positive result has more limited value (i.e., a positive test is more likely to be a false positive). Similarly, in populations at very high risk, a negative test result is more likely to be a false negative. As a result, ROC and AUC can be misleading as to the clinical utility of a test in low disease prevalence tested populations (defined as those with less than 1% rate of occurrences (incidence) per annum, or less than 10% cumulative prevalence over a specified time horizon).

In preferred embodiments, the invention relates to methods of assessing increased probability of early stage Alzheimer's disease, which can be characterized by a MMSE score of greater than 19.

Measurement of Second Indicator

Methods of assessing increased probability of Alzheimer's disease in a subject under assessment as provided herein may further include the step of obtaining a measurement of a second indicator of Alzheimer's disease in the subject under assessment. Second indicators may include, for example, other biomarkers of Alzheimer's disease, such as Aβ40 or Aβ42 monomers, Aβ42 aggregates, tau monomers and aggregates, amyloid plaques, and genetic mutations linked to Alzheimer's disease, as well as non-sample derived indicators such as age, gender, ApoE genotype, MMSE score, CDR score, Memory Box score, lifestyle factors including high blood pressure, high cholesterol, and poorly controlled diabetes, and education level.

After obtaining a measurement of a second indicator of Alzheimer's disease, the methods provided herein may include the step of calculating a subject index based on data including the Aβ40 aggregate measurement and the second indicator measurement. A subject index provides a single number that accounts for the relative contributions of data including the Aβ40 aggregate measurement and the second indicator measurement to increased probability of Alzheimer's disease. Methods of calculating the subject index will vary depending on the strength of the association of each measurement with Alzheimer's disease and any linkage between the measurements.

Any formula or model known by those of skill in the art to be useful for weighting factors contributing to a biological state or disease may be used to calculate the subject index. For example, a simple model for calculating the subject index may involve calculating a ratio between the Aβ40 aggregate measurement and the second indicator measurement. Other preferred formulas include the broad class of statistical classification algorithms, and in particular the use of discriminant analysis. The goal of discriminant analysis is to predict class membership from a previously identified set of features. In the case of linear discriminant analysis (LDA), the linear combination of features is identified that maximizes the separation among groups by some criteria. Features can be identified for LDA using an eigengene based approach with different thresholds (ELDA) or a stepping algorithm based on a multivariate analysis of variance (MANOVA). Forward, backward, and stepwise algorithms can be performed that minimize the probability of no separation based on the Hotelling-Lawley statistic.

Eigengene-based Linear Discriminant Analysis (ELDA) is a feature selection technique developed by Shen et al. (2006). The formula selects features (e.g. indicators) in a multivariate framework using a modified eigen analysis to identify features associated with the most important eigenvectors. “Important” is defined as those eigenvectors that explain the most variance in the differences among samples that are trying to be classified relative to some threshold.

A support vector machine (SVM) is a classification formula that attempts to find a hyperplane that separates two classes. This hyperplane contains support vectors, data points that are exactly the margin distance away from the hyperplane. In the likely event that no separating hyperplane exists in the current dimensions of the data, the dimensionality is expanded greatly by projecting the data into larger dimensions by taking non-linear functions of the original variables (Venables and Ripley, 2002). Although not required, filtering of features for SVM often improves prediction. Features (e.g., indicators) can be identified for a support vector machine using a non-parametric Kruskal-Wallis (KW) test to select the best univariate features. A random forest (RF, Breiman, 2001) or recursive partitioning (RPART, Breiman et al., 1984) can also be used separately or in combination to identify indicator combinations that are most important. Both KW and RF require that a number of features be selected from the total. RPART creates a single classification tree using a subset of available indicators.

A further step of the methods described herein for assessing increased probability of having Alzheimer's disease in a subject under assessment can include comparing the subject index to a control index threshold, wherein the control index threshold is calculated from data including the measurements of Aβ40 aggregates in biological samples from a plurality of cognitively normal control subjects and the measurements of a second indicator in the control subjects. The subject under assessment and the control subjects are of the same species, and all of the biological samples are of a same sample type.

Typically, the control index threshold is calculated by first calculating an index for each of the cognitively normal control subjects based on the Aβ40 aggregate measurement and the second indicator measurement for each control subject. Indices for control subjects may be calculated by the same methods described above for the subject index. Once an index has been calculated for each of the cognitively normal control subjects, a control index threshold is calculated from a model based on the indices of the control subjects. For example, the control index threshold may be calculated simply by averaging the indices of the control subjects.

In some cases, the control index threshold will be used for a diagnostic test. In such cases, the control index threshold may be calculated from a model that incorporates preferred diagnostic performance parameters, such as accuracy, sensitivity, specificity, positive predictive value, and negative predictive value. As discussed above, changing the threshold value of a test or assay usually changes the sensitivity and specificity but in a qualitatively inverse relationship. Therefore, in assessing the accuracy and usefulness of a proposed medical test, assay, or method for assessing a subject's condition, one should always take both sensitivity and specificity into account and be mindful of what the threshold is at which the sensitivity and specificity are being reported because sensitivity and specificity may vary significantly over the range of thresholds. A preferred sensitivity value for use in determining a control index threshold is at least 65%, desirably at least 70%, more desirably at least 75%, preferably at least 80%, more preferably at least 85%, and most preferably at least 90%. A preferred specificity value for use in determining a control index threshold is at least 65%, desirably at least 70%, more desirably at least 75%, preferably at least 80%, more preferably at least 85%, and most preferably at least 90%.

In some embodiments, the second indicator is measured in the same biological sample from which the Aβ aggregate measurement is obtained. In preferred embodiments, the second indicator is Aβ40 monomer or Aβ42 monomer. In particularly preferred embodiments in which the second indicator is Aβ40 monomer or Aβ42 monomer, the subject index is a ratio of Aβ40 aggregate level to the level of Aβ40 monomer or Aβ42 monomer in the subject under assessment, and the control index threshold is a control ratio threshold which is calculated from data including the levels of Aβ40 aggregates and the levels of Aβ40 monomer or Aβ42 monomer in biological samples from the control subjects. In such an embodiment, the control index threshold may be calculated by first calculating a ratio of Aβ40 aggregate level to the level of Aβ40 monomer or Aβ42 monomer in the plurality of control subjects, and subsequently calculating a control ratio threshold from a model based on the plurality of ratios. The control ratio threshold may be calculated with a model that incorporates preferred diagnostic performance parameters. This preferred embodiment may further include a step of determining that the subject under assessment has an increased probability of having Alzheimer's disease if the subject ratio is higher than the control ratio threshold. In embodiments of the methods described herein involving obtaining a measurement of a second indicator, the invention relates to methods of assessing increased probability of early stage Alzheimer's disease, which can be characterized by a MMSE score of greater than 19.

### Methods for Assessing Probability of not Having Alzheimer's Disease

According to another aspect, the invention provides methods for assessing increased probability of not having Alzheimer's disease in a subject under assessment. The methods include the steps of obtaining the level of Aβ40 aggregates in a biological sample from the subject and determining that the subject has an increased probability of not having Alzheimer's disease if the subject does not show clinical cognitive impairment and if the Aβ40 aggregate level is the same as or lower than a control level threshold. Clinical cognitive impairment is typically assessed by any clinical test known to those of skill in the art, including the Alzheimer's Disease Assessment Scale-Cognitive (ADAS-Cog), Clinical Dementia Rating (CDR), Memory Box score, and the Mini-Mental State Examination (MMSE). Clinical cognitive impairment can be indicated by outcomes from such clinical tests as those known to those of skill in the art. The control level threshold is calculated from data including the levels of Aβ40 aggregates in biological samples from a plurality of cognitively normal control subjects. The subject under assessment and the control subjects are of the same species, and all of the biological samples are of the same sample type and do not include brain tissue, fractions of brain tissue, or brain homogenate.

According to yet another aspect, the invention provides methods for assisting in assessing increased probability of not having Alzheimer's disease in a subject under assessment. The methods include the steps of measuring the level of Aβ40 aggregates in a biological sample from the subject and communicating the Aβ40 aggregate level to a different entity, wherein the different entity determines that the subject has an increased probability of not having Alzheimer's disease if the subject does not show clinical cognitive impairment and if the Aβ40 aggregate level is the same as or lower than a control level threshold. The control level threshold is calculated from data including the levels of Aβ40 aggregates in biological samples from a plurality of cognitively normal control subjects. The subject under assessment and the control subjects are of the same species, and all of the biological samples are of the same sample type and do not include brain tissue, fractions of brain tissue, or brain homogenate.

According to other aspects, methods for assessing increased probability of not having Alzheimer's disease or for assisting in assessing increased probability of not having Alzheimer's disease may include the step of obtaining or measuring a ratio of Aβ40 aggregate level to Aβ42 monomer level in a biological sample taken from the subject.

### Methods for Monitoring Disease Progression

The invention described herein provides methods for monitoring disease progression in a subject with Alzheimer's disease. These methods are based on the further discovery described in the Examples that levels of Aβ40 aggregates and the ratios of Aβ40 aggregate level to Aβ42 monomer level in a subject with Alzheimer's disease decrease as the disease progresses. According to the methods for monitoring disease progression as described herein, the subject with Alzheimer's disease has not undergone any treatment either currently known to be a treatment or shown in the future to be a treatment for Alzheimer's disease. According to this and other aspects of the invention, current treatments for Alzheimer's disease include, for example, cholinesterase inhibitors such as donepezil (Aricept), rivastigmine (Exelon) and galantamine (Razadyne), NMDA glutamate receptor blockers such as memantine (Namenda), and herbal remedies such as Huperzine A. Treatments currently under development include immunotherapy and Aβ vaccines.

The invention provides methods for monitoring disease progression in a subject with Alzheimer's disease who has not undergone any treatment for Alzheimer's disease including the first step of obtaining the level of Aβ40 aggregates in a biological sample taken from the subject at a time after the subject is diagnosed with Alzheimer's disease. The methods then include the step of determining that the subject has an increased probability of having progressed to an advanced stage of Alzheimer's disease if (a) the Aβ40 aggregate level is lower than an early stage level of Aβ40 aggregates in the subject if available, or if (b) the Aβ40 aggregate level is lower than an early stage standard if the early stage level of Aβ40 aggregates in the subject is not available. Alternatively, the methods include the step of determining that the subject has a decreased probability of having progressed to an advanced stage of Alzheimer's disease if (a) the Aβ40 aggregate level is the same as the early stage level of Aβ40 aggregates in the subject if available, or if (b) the Aβ40 aggregate level is the same as the early stage standard if the early stage level of Aβ aggregates in the subject is not available. The early stage level can be the level of Aβ40 aggregates in a biological sample taken from the subject at early stage of Alzheimer's disease, or the level of Aβ40 aggregates in one or more biological samples taken from one or more standard subjects with known early stage of Alzheimer's disease. The subject and the standard subjects are of the same species, and all of the biological samples of a same sample type and do not include brain tissue, a fraction of brain tissue, or brain homogenate.

In another aspect, the invention provides methods for assisting in monitoring disease progression in a subject with Alzheimer's disease who has not undergone any treatment for Alzheimer's disease. The methods include the steps of measuring the level of Aβ40 aggregates in a biological sample taken from the subject at a time after the subject is diagnosed with Alzheimer's disease and communicating the Aβ40 aggregate level to a different entity. The different entity then determines that the subject has an increased probability of having progressed to an advanced stage of Alzheimer's disease if (a) the Aβ40 aggregate level is lower than an early stage level of Aβ40 aggregates in the subject if available, or if (b) the Aβ40 aggregate level is lower than an early stage standard if the early stage level of Aβ40 aggregates in the subject is not available. Alternatively, the different entity then determines that the subject has a decreased probability of having progressed to an advanced stage of Alzheimer's disease if (a) the Aβ40 aggregate level is the same as the early stage level of Aβ40 aggregates in the subject if available, or if (b) the Aβ40 aggregate level is the same as the early stage standard if the early stage level of Aβ aggregates in the subject is not available. The early stage level can be the level of Aβ40 aggregates in a biological sample taken from the subject at early stage of Alzheimer's disease, or the level of Aβ40 aggregates in one or more biological samples taken from one or more standard subjects with known early stage of Alzheimer's disease. The subject and the standard subjects are of the same species, and all of the biological samples are of a same sample type and do not include brain tissue, fractions of brain tissue, or brain homogenate.

According to other aspects, methods for monitoring disease progression in a subject with Alzheimer's disease or for assisting in monitoring disease progression in a subject with Alzheimer's disease may include the step of obtaining or measuring a ratio of Aβ40 aggregate level to Aβ42 monomer level in a biological sample taken from the subject at a time after the subject is diagnosed with Alzheimer's disease.

Similar to the methods of determining a threshold as described in the above section, the early stage standard may be calculated by any models known by one of skill in the art to manipulate the levels of Aβ40 aggregates in biological samples taken from a plurality of standard subjects with early stage of Alzheimer's disease to generate an average or other meaningful numerical summary of the levels. In certain embodiments, the early stage standard is the mean of levels of Aβ40 aggregates in biological samples taken from a plurality of standard subjects with early stage of Alzheimer's disease.

Early stage of Alzheimer's disease, as used herein, is characterized by an MMSE score of greater than 19.

### Methods for Assigning Disease Stage

The invention described herein also provides methods for assigning disease stage for a subject with Alzheimer's disease prior to any treatment for Alzheimer's disease. The methods include the steps of obtaining the level of Aβ40 aggregates in a biological sample taken from the subject and determining that the subject has an increased probability of having the same disease stage as that of a stage-specific standard, if available, if the Aβ40 aggregate level is close to the standard. Typically, the Aβ40 aggregate level is considered to be “close” to the standard if the level of Aβ40 aggregate is within a 95% confidence interval of the standard. 95% confidence limits are defined by the mean±2(standard deviation) for a normal population. The standard is the level of Aβ40 aggregates in one or more biological samples taken from one or more standard subjects with a same known stage of Alzheimer's disease. The subject and the standard subjects are of the same species, and all of the biological samples are of a same sample type and do not include brain tissue, fractions of brain tissue, or brain homogenate.

In another aspect, the invention provides methods for assisting in assigning disease stage for a subject with Alzheimer's disease prior to any treatment for Alzheimer's disease. The methods include the steps of measuring the level of Aβ40 aggregates in a biological sample taken from the subject and communicating the Aβ40 aggregate level to a different entity. The different entity then determines that the subject has an increased probability of having the same disease stage as that of a standard, if available, if the Aβ40 aggregate level is close to the standard. The standard is the level of Aβ40 aggregates in one or more biological samples taken from one or more standard subjects with a same known stage of Alzheimer's disease. The subject and the standard subjects are of the same species, and all of the biological samples are of a same sample type and do not include brain tissue, fractions of brain tissue, or brain homogenate.

According to other aspects, methods for assigning disease stage for a subject with Alzheimer's disease prior to treatment for Alzheimer's disease or for assisting in assigning disease stage in a subject with Alzheimer's disease prior to treatment with Alzheimer's disease may include the step of obtaining or measuring a ratio of Aβ40 aggregate level to Aβ42 monomer level in a biological sample taken from the subject.

According to the invention, disease stage refers to any stage of Alzheimer's disease known to one of skill in the art. For example, disease stage includes early stage of Alzheimer's disease and advanced stage of Alzheimer's disease. Early stage of Alzheimer's disease, as used herein, is characterized by an MMSE score of greater than 19 and includes MCI. Advanced stage of Alzheimer's disease, as used herein, is characterized by an MMSE score of 19 and below and includes progressive (MMSE=15-19) and late stage (MMSE<15) Alzheimer's disease. Standard subjects may be classified as having a specific stage of Alzheimer's disease according to methods known to one of skill in the art.

Similar to the methods of determining a threshold as described in the above section, the stage-specific standard may be calculated by any models known by one of skill in the art to manipulate the levels of Aβ40 aggregates in biological samples taken from a plurality of standard subjects with a known stage of Alzheimer's disease to generate an average or other meaningful numerical summary of the levels. In certain embodiments, the standard is the mean of levels of Aβ40 aggregates in biological samples taken from a plurality of standard subjects with a same known stage of Alzheimer's disease. In certain embodiments, at least one early-stage standard and one advanced stage standard are available for comparison with the Aβ40 aggregate level.

### Methods for Assessing Increased Probability of MCI Progressing to Alzheimer's Disease

According to yet another aspect, the invention provides methods for prognosis such as assessing increased probability of MCI progressing to Alzheimer's disease in a subject under assessment. The methods include the steps of obtaining a measurement of Aβ40 aggregates in a biological sample from a subject and determining that the subject has an increased probability of MCI progressing to Alzheimer's disease if the Aβ40 aggregate level is higher than a control level threshold. The control level threshold is calculated from data including the levels of Aβ40 aggregates in biological samples from a plurality of cognitively normal control subjects. The subject under assessment and the control subjects are of a same species; and all of the biological samples are of the same sample type, preferably a CSF sample, and wherein none of the biological samples include brain tissue, a fraction of brain tissue or brain homogenate.

In a related aspect, the invention provides method for assisting in assessing increased probability of MCI progressing to Alzheimer's disease in a subject under assessment. The methods include the steps of measuring the level of Aβ40 aggregates in a biological sample from a subject; and communicating the Aβ40 aggregate level to a different entity; wherein the different entity determines that the subject has an increased probability of MCI progressing to Alzheimer's disease if the Aβ40 aggregate level is higher than a control level threshold. The control level threshold is calculated from data including the levels of Aβ40 aggregates in biological samples from a plurality of cognitively normal control subjects. The subject under assessment and the control subjects are of a same species; and all of said biological samples are of the same sample type, preferably a CSF sample and none of the biological samples include brain tissue, a fraction of brain tissue or brain homogenate.

According to other aspects, method for assessing increased probability of MCI progressing to Alzheimer's disease may include the step of obtaining or measuring a ratio of Aβ40 aggregate level to Aβ42 monomer level in a biological sample taken from the subject.

Methods of assessing increased probability of MCI progressing to Alzheimer's disease in a subject under assessment as provided herein may further include the step of obtaining a measurement of a second indicator of MCI progressing to Alzheimer's disease in the subject under assessment. Second indicators may include, for example, other biomarkers of Alzheimer's disease, such as Aβ40 or Aβ42 monomers, Aβ42 aggregates, tau monomers and aggregates, amyloid plaques, and genetic mutations linked to Alzheimer's disease, as well as non-sample derived indicators such as age, gender, ApoE genotype, MMSE score, CDR score, Memory Box score, lifestyle factors including high blood pressure, high cholesterol, and poorly controlled diabetes, and education level.

After obtaining a measurement of a second indicator of MCI progressing to Alzheimer's disease, the methods provided herein may include the step of calculating a subject index based on data including the Aβ40 aggregate measurement and the second indicator measurement and methods of calculating a subject index as described in the section regarding “Measurement of a Second Indicator” for “Methods for Assessing Increased Probability of Having Alzheimer's Disease”.

A further step of the methods described herein for assessing increased probability of MCI progressing to Alzheimer's disease in a subject under assessment can include comparing the subject index to a control index threshold, wherein the control index threshold is calculated from data including the measurements of Aβ40 aggregates in biological samples from a plurality of cognitively normal control subjects and the measurements of a second indicator in the control subjects. Control indexes can be calculated as described in the section regarding “Measurement of a Second Indicator” for “Methods for Assessing Increased Probability of Having Alzheimer's Disease”.

### Subjects

In preferred embodiments of the invention, the subjects are humans. In other embodiments, the subjects are non-human animals. Examples of preferred non-human animals include mice.

In additional preferred embodiments of the invention, the subject under assessment and the subject with Alzheimer's disease may be alive. In further preferred embodiments of the invention, the control subjects and the standard subjects may be alive.

### Biological Samples

Biological samples used in the methods of the invention do not include brain tissue, fractions of brain tissue, or brain homogenate. Preferred biological samples may include bodily fluid or bodily tissue. In certain embodiments, Aβ40 aggregates are circulating. Preferred biological samples also include whole blood, blood fractions, blood components, plasma, platelets, serum, cerebrospinal fluid (CSF), bone marrow, urine, tears, milk, lymph fluid, organ tissue, nervous system tissue, non-nervous system tissue, muscle tissue, biopsy, necropsy, fat biopsy, fat tissue, cells, feces, placenta, spleen tissue, lymph tissue, pancreatic tissue, bronchoalveolar lavage (BAL), or synovial fluid. Particularly preferred biological samples include plasma, serum, CSF, and urine. In other embodiments, the biological sample includes CSF.

### III. Practicing Methods of the Invention

The steps required to achieve the objects of the invention may be practiced by either one or more than one entity. An entity may be, for example, a person, a group of people, an institution, or a business.

### One Entity

According to certain aspects, the steps required to achieve the objects of the invention are practiced by one entity. In certain embodiments, a single entity may obtain the levels of indicators in biological samples from a subject and determine that the subject has an increased probability of having or not having Alzheimer's disease, has an increased or decreased probability of having progressed to an advanced stage of Alzheimer's disease, or has an increased probability of having the same disease stage as that of a standard. This entity may be, for example, a doctor or a clinician. This entity may also be an institution, such as a hospital or a doctor's office, wherein all steps of the methods are performed by employees of the institution. “Obtain” as used herein can include measuring, receiving, or other ways of obtaining information either directly or indirectly. In aspects of the invention wherein an entity obtains a ratio, obtaining a ratio can include calculating from the measurements or levels after a measurement step performed by the same or different entity, receiving a ratio, or other ways of obtaining a ratio either directly or indirectly.

In yet other embodiments, a single entity may measure the levels of indicators in biological samples from a subject and communicate the indicator levels to a different entity that then determines that the subject has an increased probability of having or not having Alzheimer's disease, has an increased or decreased probability of having progressed to an advanced stage of Alzheimer's disease, or has an increased probability of having the same disease stage as that of a standard. In these embodiments, the single entity performing the steps of measuring and communicating may be, for example, a clinic or a lab technician.

More than One Entity

According to other aspects, the steps required to achieve the objects of the invention may be performed by more than one entity. One entity may measure the levels of indicators in biological samples from a subject, whereas a different entity may determine that the subject has an increased probability of having or not having Alzheimer's disease, has an increased or decreased probability of having progressed to an advanced stage of Alzheimer's disease, or has an increased probability of having the same disease stage as that of a standard. For example, a lab technician at a clinic may measure the levels of indicators in biological samples from a subject, and a doctor at a hospital may determine that the subject has an increased probability of having or not having Alzheimer's disease, has an increased or decreased probability of having progressed to an advanced stage of Alzheimer's disease, or has an increased probability of having the same disease stage as that of a standard.

### Communication Between Entities

Communication between entities in practicing methods of the invention may occur by any means used in the art. Typically, information communicated between entities will be in the form of a report. In certain embodiments, a first entity may obtain levels of indicators in biological samples from a second entity that measures the levels of indicators in biological samples. The first entity may obtain the levels from the second entity directly or indirectly. For example, the first entity may obtain a paper or electronic report of the levels directly from the second or different entity. In another example, the first entity may obtain an electronic report of the levels on a network to which the second entity has uploaded the levels. In yet another example, the first entity may obtain the levels from a third entity that has prepared a report from the measurements made by the second entity.

In other embodiments, a first entity may measure the levels of indicators in biological samples and communicate the levels to a second or different entity that then determines that the subject has an increased probability of having or not having Alzheimer's disease, has an increased or decreased probability of having progressed to an advanced stage of Alzheimer's disease, or has an increased probability of having the same disease stage as that of a standard. The first entity may communicate the levels to the second entity directly or indirectly. For example, the first entity may prepare a report with the levels and give the report to the second entity manually or electronically. In another example, the first entity may upload the levels to a network from which the second entity can obtain the levels. In yet another example, the first entity may communicate the levels to a third entity that prepares a report for use by the second entity.

### Clinical Trials

Methods of the invention may be useful for clinical trials involving Alzheimer's disease. Typically, for their use in clinical trials, the steps required to achieve the objects of the invention will be practiced by more then one entity. For example, multiple entities in different locations, such as clinics in different cities, may measure the levels of indicators in biological samples from subjects under assessment and communicate the levels to a different entity, such as a group of doctors directing the clinical trial, who then determine that the subject has an increased probability of having or not having Alzheimer's disease, has an increased or decreased probability of having progressed to an advanced stage of Alzheimer's disease, or has an increased probability of having the same disease stage as that of a standard. In certain embodiments, the multiple entities may use a third entity that prepares and communicates reports of the levels to the different entity that determine that the subject has an increased probability of having or not having Alzheimer's disease, has an increased or decreased probability of having progressed to an advanced stage of Alzheimer's disease, or has an increased probability of having the same disease stage as that of a standard. Clinical trials involving methods of the invention may be useful for evaluating effectiveness of new drugs and treatments for Alzheimer's disease or for evaluating the effect of various clinical parameters on Alzheimer's disease and its progression.

### IV. Obtaining Measurements and Levels of Aβ40 Aggregates in Methods of the Invention

Methods of the invention include the step of obtaining a measurement or level of Aβ40 aggregates in a biological sample from a subject under assessment. Obtaining a measurement or level of Aβ40 aggregates may be accomplished by any methods known to one of skill in the art such as Misfolded Protein Assay (MPA) (Lau et al., 2007, PNAS, 104: 11551), ELISA, seeded multimerization, nanoparticle based detection, nanoscale optical biosensor method, dual color FRET detection with flow cytometry, and dual color single aggregate FCS detection (Funke et al., Current Alzheimer Research, 2009, 6: 285-289). Exemplary reagents useful for obtaining a measurement or level of Aβ40 aggregate include mAb158 (Englund et al. J. Neurochem 2007, 103: 334-345) and antibodies described by Kayed et al. (Science, 2003, 300: 486-489 and Mol. Neurodegeneration, 2007, 2:18), which bind specifically to Aβ aggregates. Other antibodies known to those of skill in the art may also be used.

In certain embodiments, the Aβ40 aggregate level is obtained by a method including a first step of contacting bodily fluid or a homogenate of bodily tissue with an aggregate-specific binding reagent under conditions that allow binding of the reagent to Aβ40 aggregates, if present, to form a complex. Aggregate-specific binding reagents may be any reagent, such as a peptoid, a peptide, or a dendron, that binds preferentially to aggregate over monomer when attached to a solid support at certain charge densities. A reagent is said to “bind preferentially” to an aggregate if it binds with greater affinity, avidity, and/or greater specificity to the aggregate than to monomer. Aggregate-specific binding reagents may include, for example, those described in U.S. Provisional Patent Application No. 61/258,188, which is hereby incorporated by reference. These aggregate-specific binding reagents may be detectably labeled with labels including, without limitation, tags (e.g., biotin, His-Tags, oligonucleotides), dyes, fluorophores, and members of a binding pair.

The aggregate-specific binding reagent is attached to a solid support. Solid supports to be used include, without limitation, nitrocellulose, polystyrene latex bead, titanium oxide, silicon oxide, polysaccharide bead, polysaccharide membrane, agarose, glass, polyacrylic acid, polyethyleneglycol, polyethyleneglycol-polystyrene hybrid, controlled pore glass, glass slide, gold bead, and cellulose. Aβ40 aggregate-specific binding reagents may be attached to solid supports by any methods known to one of skill in the art.

Methods of obtaining measurements or levels of Aβ40 aggregate also include a subsequent step of detecting Aβ40 aggregates, if any, in the biological sample by their binding to the aggregate-specific binding reagent. In certain embodiments, the detecting step of the methods may include the substeps of separating the complex formed by the reagent and the Aβ40 aggregates from unbound monomers of Aβ40, if present, optionally, dissociating Aβ40 aggregates from the complex, and detecting Aβ40 aggregate. In other embodiments, the detecting step of the methods may include the substeps of separating the complex formed by the reagent and Aβ40 aggregates from unbound monomers of Aβ40, if present, and removing the unbound monomers of Aβ40, denaturing the Aβ40 aggregates present in the complex to form Aβ40 monomers, and detecting Aβ40 monomers. Separating and removing the complex formed by the reagent and Aβ40 aggregates from unbound monomers of Aβ40 can be achieved by immunoprecipitation and washes, by size exclusion chromatography, or by any other methods known to one of skill in the art. “Dissociation” refers to the physical separation of the Aβ40 aggregate from the reagent such that the aggregate can be detected separately from the reagent. Dissociation and denaturation of the Aβ40 aggregate from the complex can be accomplished, for example, using 3.0 to 6.0 M of guanidinium hydrochloride or guanidinium isothiocyanate. Aβ40 aggregates may also be dissociated and denatured to form Aβ40 monomers by any methods known to one of skill in the art, such as by altering pH.

After the complex is separated by unbound monomers, if present, the Aβ40 aggregates may be detected by a detection reagent. Detection reagents may be any reagent, typically an antibody, that binds specifically to Aβ40 aggregates and/or Aβ40 monomers. Suitable detection reagents are described in U.S. Provisional Patent Application No. 61/258,188. Preferred detection reagents include 11A50-B10 (Covance), an antibody specific for C-terminus of Aβ40; 4G8, specific for Aβ amino acids 18-22; 20.1, specific for Aβ amino acids 1-10; and 6E10, specific for Aβ amino acids 3-8. In certain embodiments, the detection reagent is detectably labeled by labels including, without limitation, tags (e.g., biotin, His-Tags, oligonucleotides), dyes, fluorophores, and members of a binding pair.

The following non-limiting examples are described for illustration.

### EXAMPLES

### Example 1

### Aβ40 Aggregates are Detected in CSF Samples from Alzheimer's Disease Patients Using the Misfolded Protein Assay (MPA)

This Example shows for the first time that circulating Aβ40 aggregates can be detected in a biological sample, in particular in a biological fluid sample of CSF. More importantly, this Example shows that CSF samples from Alzheimer's disease patients have higher levels of Aβ40 aggregates than control samples from people who do not have Alzheimer's disease and are presumed to be cognitively normal. The Misfolded Protein Assay (MPA) using capture reagent PSR1 (peptoid reagent XIIb attached to magnetic beads) was used to selectively detect Aβ40 aggregates as opposed to Aβ40 monomers.

The structure of PSR1 is provided below:

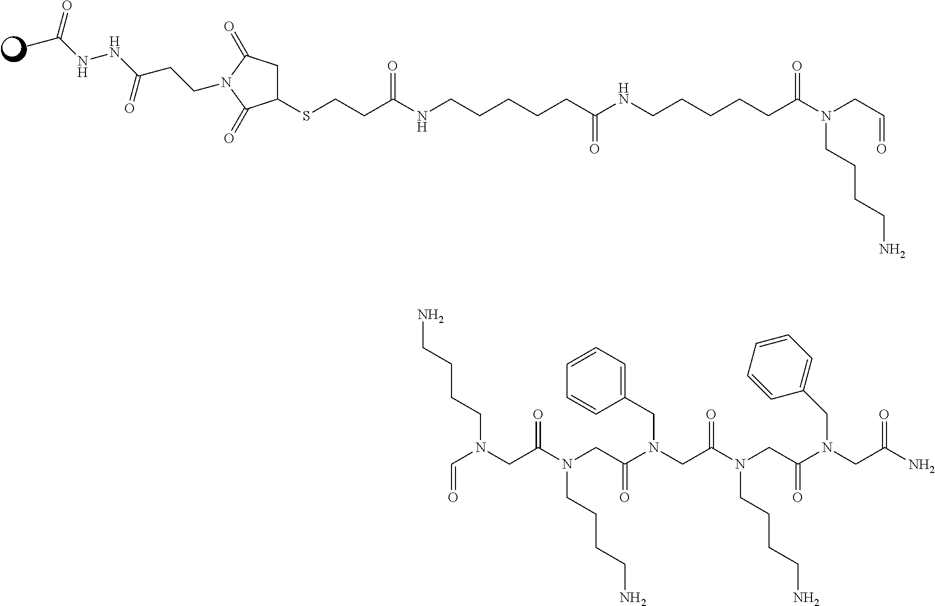 [SMILES: CC(=O)NNC(=O)CCN1C(=O)CC(SCCC(=O)NCCCCCC(=O)NCCCCCC(=O)N(CC=O)CCCCN)C1=O ; NCCCCN(C=O)CC(=O)N(CCCCN)CC(=O)N(CC(=O)N(CCCCN)CC(=O)N(CC(N)=O)Cc1ccccc1)Cc1ccccc1]

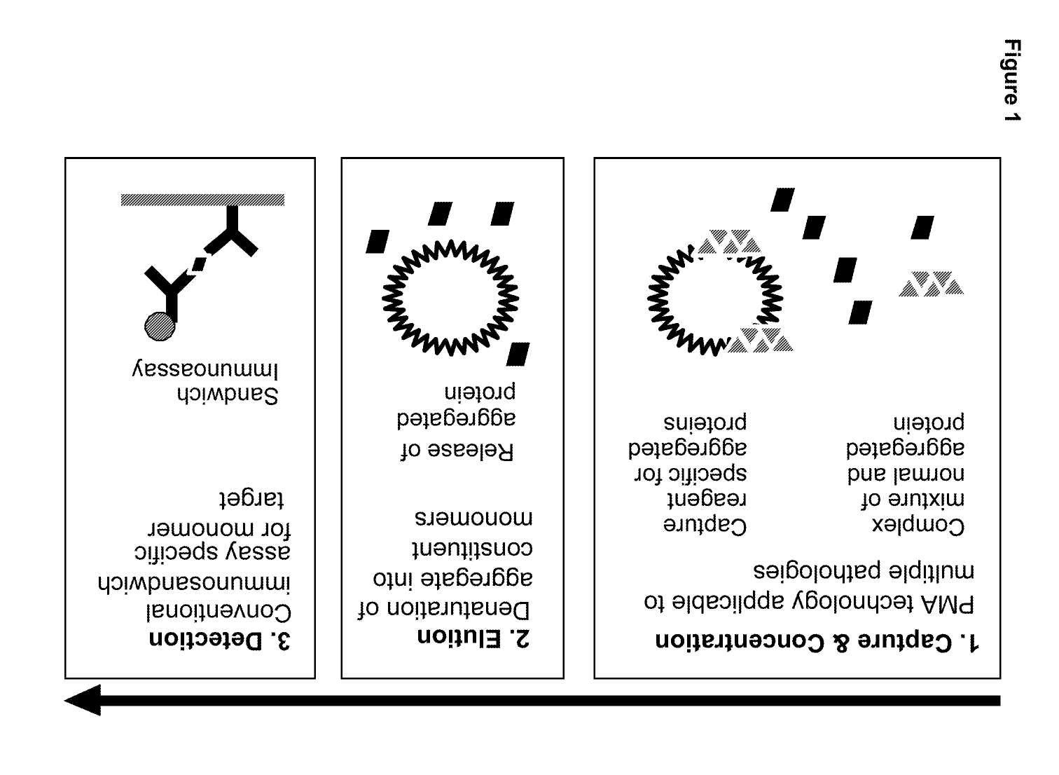

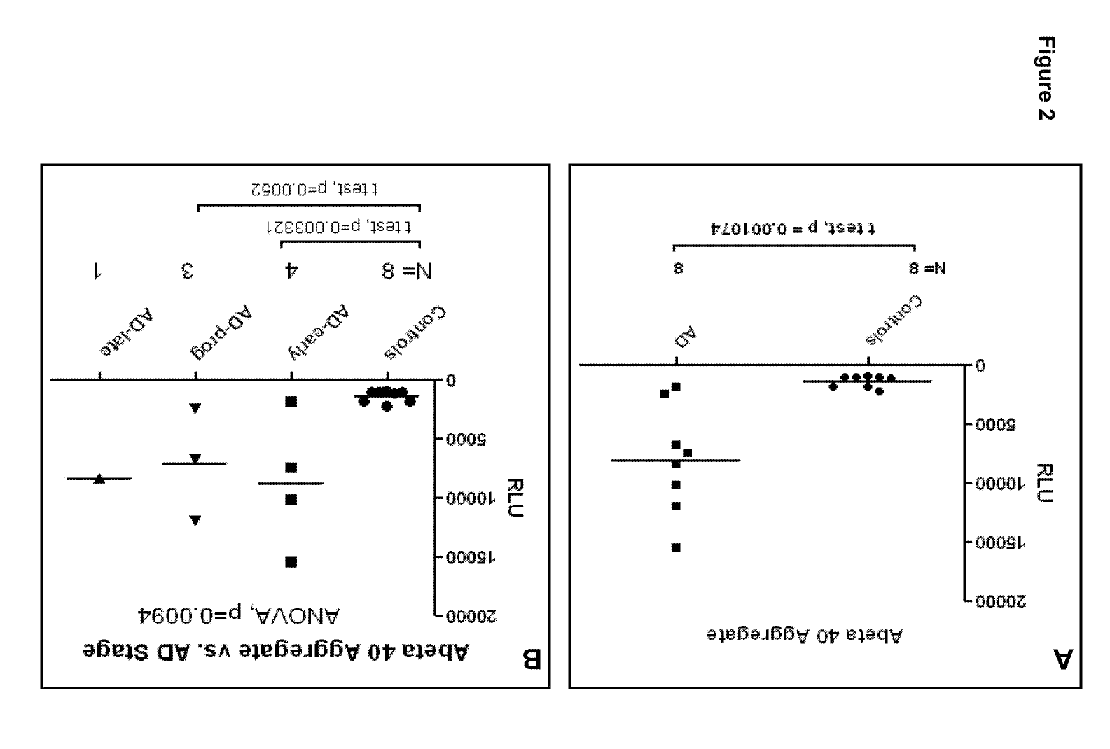

A schematic of the basic MPA is shown in FIG. 1. In brief, there is an initial amyloid-selective bead-based capture step followed by elution and specific detection of particular amyloid protein species. A minimum of 200 microliters of patient CSF is currently used for each data point.

In this Example, CSF samples from eight Alzheimer's disease patients (or AD samples) and eight normal control individuals were obtained from Analytical Biological Sciences (ABS). The eight AD samples were classified into disease stages based on clinical cognitive mini mental state examination (MMSE) test scores. The following criteria were used for grouping the samples tested in this Example.

| Stage | Substage | MMSE score |
|---|---|---|
| Early stage | | >19 |
| Advanced stage | Progressive AD | 15-19 |
| | Late AD | <15 |

200 μl of each of these CSF samples was incubated with 50 μl of 5×TBSTT (250 mM Tris, pH 7.5; 750 mM NaCl; 5% Tween-20; 5% Triton X-100) and 30 μl of PSR1 (peptoid reagent XIIb covalently bound to Dynal M270-carboxylic acid beads at 30 mg/mL) at 37° C. for 1 hour with shaking at 550 rpm.

A negative control for the assay was carried out using 250 μl/assay of 1×TBSTT buffer (50 mM Tris, pH 7.5; 150 mM NaCl; 1% Tween-20; 1% Triton X-100). in triplicate. A positive control was carried out using 125 μl/assay of sonicated 100 nl 5% ADBH (Alzheimer's disease brain homogenate) in 200 μl normal CSF (CSF from individuals without Alzheimer's disease) and 50 μl of 1×TBSTT.

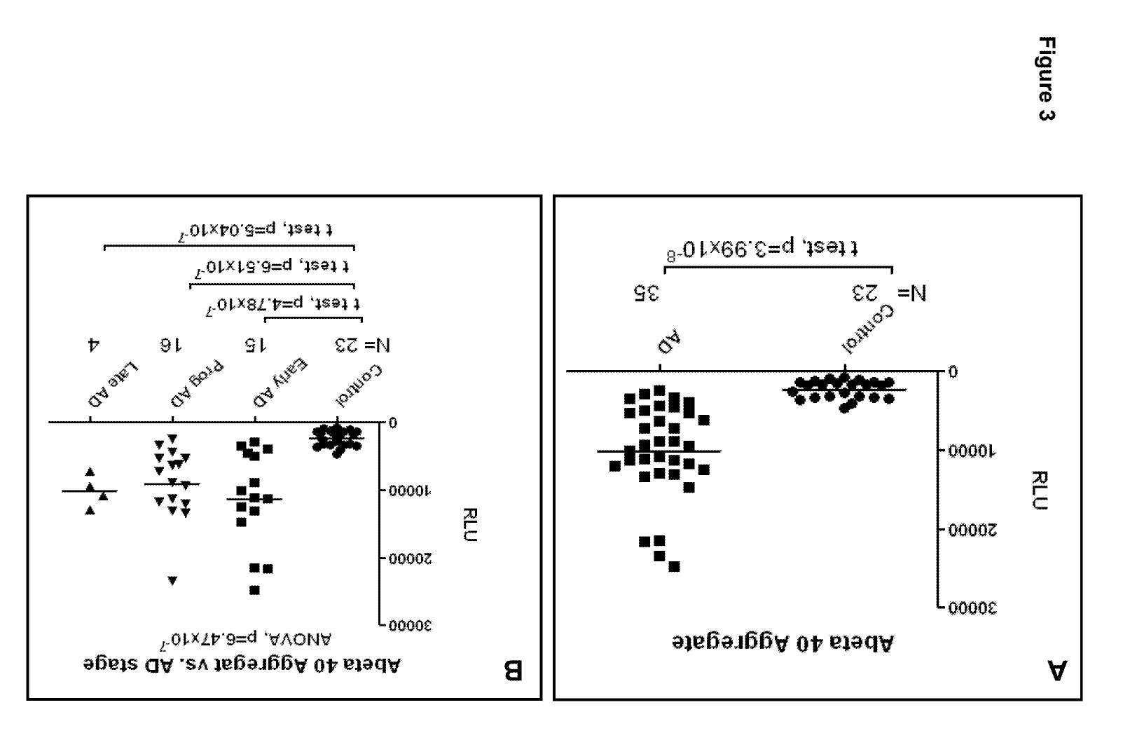

The beads were washed eight times with TBST (50 mM Tris, pH 7.5; 150 mM NaCl; 0.05% Tween-20) using magnetic separation to immobilize the beads while the wash buffer was removed. 100 μl of 1% Zwittergent 3-14 (from Sigma, product number T7763) was added and the mixture was incubated at room temperature for 30 minutes with shaking at 750 rpm. The beads were washed again 8 times with TBST using magnetic separation. Aggregates were eluted and denatured by adding 20 μl of 0.1 N NaOH and incubating at 80° C. for 30 minutes with shaking at 750 rpm. The assay plate was immediately put on ice for one minute, then neutralized with the addition of 20 μl of 0.12 M NaH2PO4 containing 0.4% Tween-20. The mixture was incubated for 5 minutes at room temperature with shaking at 750 rpm. Magnetic separation was applied to the assay plate and the supernatants were transferred to an ELISA plate which is a component of a kit from Meso Scale Discovery (Gaithersburg, Md.), the MSD® 96-Well MULTI-SPOT® Human/Rodent [4G8] Abeta Triplex Ultra-Sensitive Assay. ELISA was carried out according to the protocol of the kit which allows for multiplex detection of Aβ38, Aβ40 and Aβ40. The immunoassay results for Aβ40 detection obtained with the kit is shown in FIG. 2.

FIG. 2A shows that CSF samples from Alzheimer's disease patients have statistically significant higher levels of Aβ40 aggregates than the non-diseased and presumed to be cognitively normal control samples.

In FIG. 2B the AD sample groups are separated according to disease stages. ANOVA test shows that the AD samples and the control samples do not come from a single population. Individual t-tests indicate a significant difference between the early stage AD sample group and the control group, as well as between the progressive substage AD sample group and the control group. Due to the low number of samples in this study, diagnostic sensitivity and specificity were not calculated.

### Example 2

### Aβ40 Aggregate Levels are Increased in CSF Samples from Patients in Early and Advanced Stages of Alzheimer's Disease

This Example confirms that elevated levels of Aβ40 aggregates can be detected in CSF samples from patients in early and advanced stages of Alzheimer's disease and suggests that early stage patients have a higher CSF Aβ40 aggregate level than patients in advanced stage Alzheimer's disease.

In this Example, CSF samples from 35 AD and 23 control samples were obtained from ABS. These samples were grouped into early stage and progressive and late AD substages as defined by ABS. MMSE scores were not available for these samples so confirmation of classification as described for Example 1 was not possible. The samples were tested using the same methods described in Example 1.

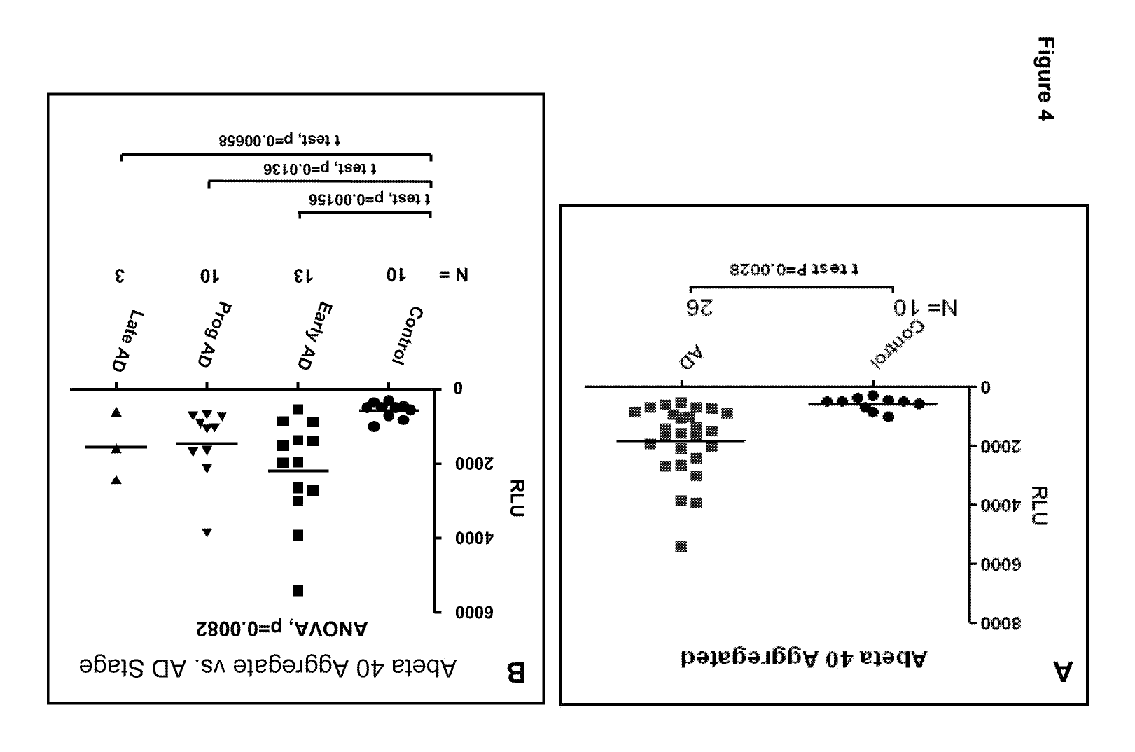

FIG. 3A confirms that CSF samples from Alzheimer's disease patients have statistically significant higher levels of Aβ40 aggregates than the non-diseased and presumed to be cognitively normal control samples.

In FIG. 3B the AD sample groups are separated according to stage and substages as classified by ABS. ANOVA test shows that the AD samples and the control samples do not come from a single population. Individual t-tests show a significant difference between the ABS early stage AD sample group and the control group, between the ABS progressive substage AD sample group and the control group, and between the ABS late substage sample group and the control group.

A clinical sensitivity of 82.86% and clinical specificity of 95.65% for AD was observed with a decision threshold of mean control group RLU value plus 2 standard deviations.

### Example 3

### Clinical CSF Samples Obtained from Another Source Confirm that Aβ40 Aggregate Levels are Increased in Patients in Early and Advanced Stages of Alzheimer's Disease and that Aβ40 Aggregate is a Biomarker for AD and Early Stage AD

This Example shows that elevated levels of Aβ40 aggregates can be detected in CSF samples from patients in early and advanced stages of Alzheimer's disease and that Aβ40 aggregate level is a useful biomarker for AD and early stage AD.

In this Example, 26 AD and 10 control samples were grouped into different disease stages and substages using the criteria discussed in Example 1 and tested using the same method as described in Example 1.

CSF samples were obtained from the university research hospital. The AD group consisted of 26 patients consulting the memory disorder clinic, mean age 71.8±7.3 years. Patients diagnosed with AD had to meet the DSM-IIIR criteria of dementia [American Psychiatric Association (1987) Diagnostic and Statistical Manual of Mental Disorders, Third edition revised. American Psychiatric Association, Washington, D.C., USA] and the criteria of probable AD defined by NINCDS-ADRDA [McKann G, Drachman D, Folsyein M, Katzman R, Pricxe D, Stadlan E M (1984) Clinical diagnosis of Alzheimer's disease: report of the NINCDS-ARADA Work Group under the auspices of Department of Health Service Task Force on Alzheimer's Disease. Neurology 34, 939-944]. The AD patients were followed over time with additional clinical evaluations. The control group, in total 10 cases, mean age 69.4±9.7 years, was defined based on absence of memory complaints or any other cognitive symptoms, and no signs of active neurological or psychiatric disease. All patients and controls gave informed consent to participate in the study. CSF was collected in polypropylene tubes, centrifuged, aliquoted, and stored at −80° C. pending analyses.

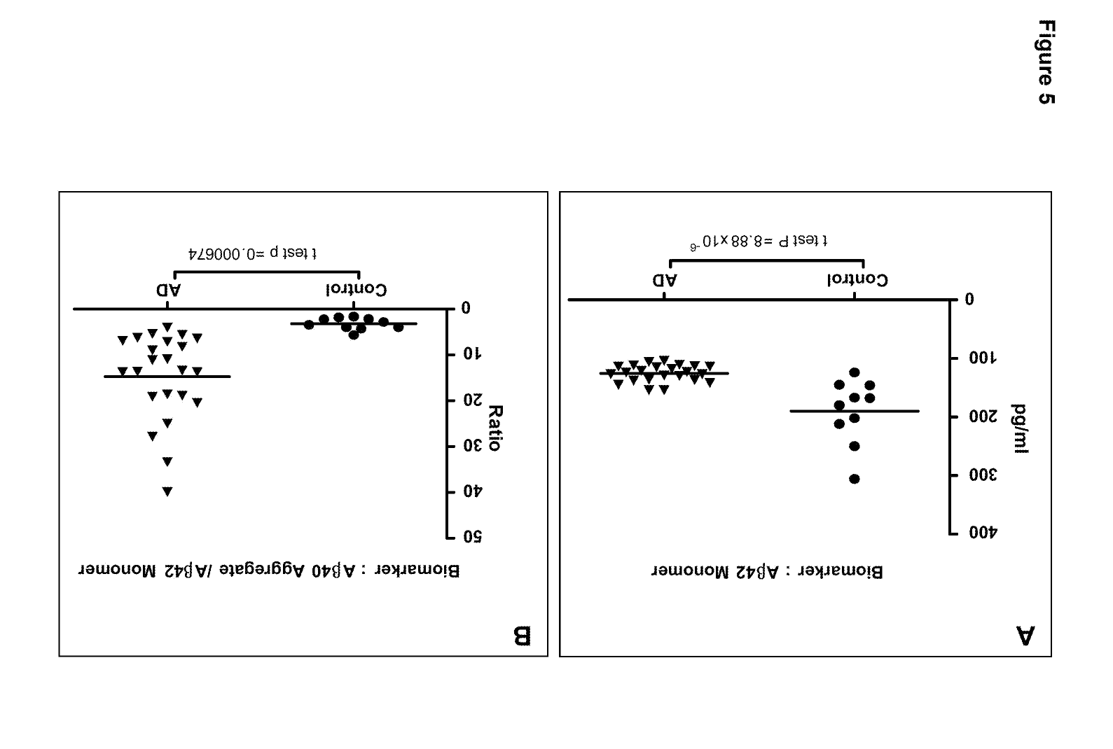

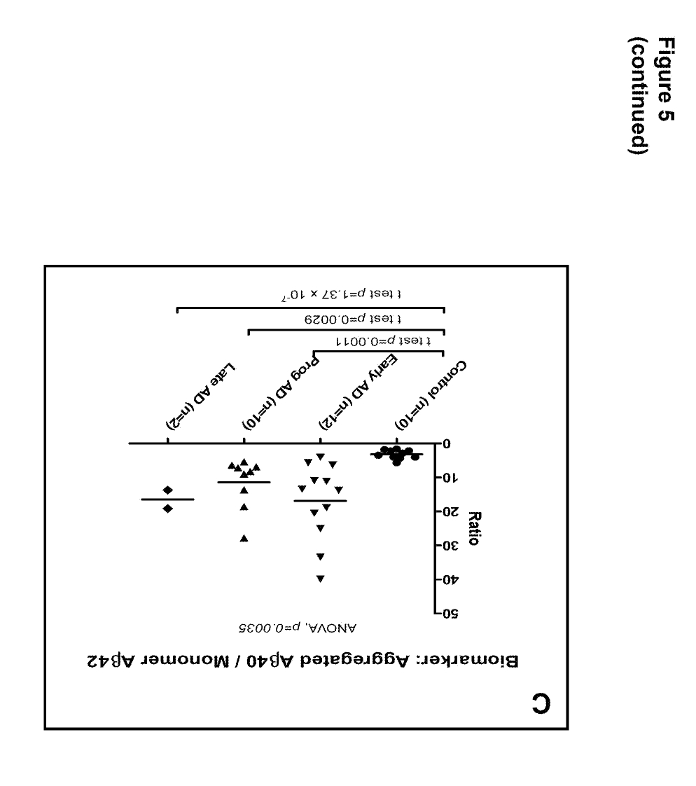

FIG. 4A confirms using a different set of clinical CSF samples that Alzheimer's disease patients have higher levels of Aβ40 aggregates than non-diseased and presumed to be cognitively normal control samples.

In FIG. 4B, the AD sample groups are separated according to disease stages and substages. ANOVA test shows that the AD samples and the control samples do not come from a single population. Individual t-tests show a significant difference between the early stage AD sample group and the control group, between the progressive substage AD sample group and the control group, and between the late substage AD sample group and the control group.

ROC analysis was conducted to determine the optimal sensitivity and specificity for Alzheimer's disease. Graph Pad Prism version 5.0 was used to conduct the ROC analysis. This program generates an ROC curve and the associated AUC, Sensitivity, and Specificity values for a number of different threshold/cutoff values. The optimal threshold/cutoff value which provides for the best sensitivity and specificity is then manually determined by simply choosing the threshold value which provides maximal sensitivity and specificity (i.e. the smallest difference between these two parameters).

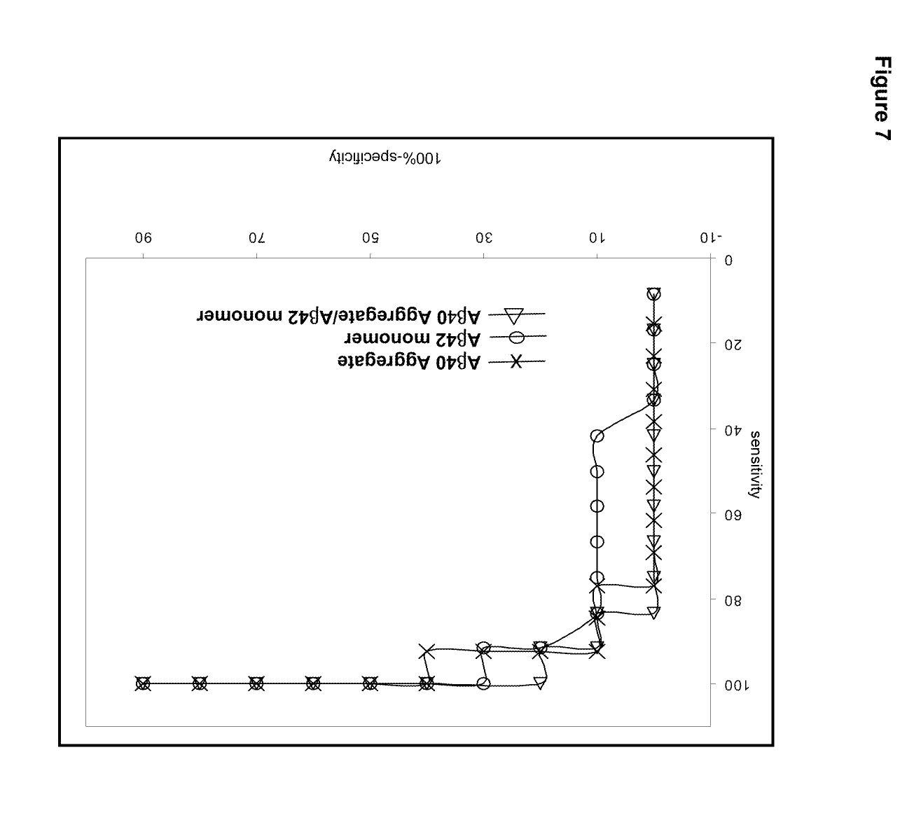

The ROC curve is shown in FIG. 6. The diagnostic performance parameters for Aβ40 aggregate are provided below.

As used herein, parameters are as defined above. Test accuracy is calculated by (TP+TN)/(TP+TN+FP=FN).

| Parameters | Aβ40 Aggregate (MPA) |
|---|---|
| ROC AUC | 0.9307 |
| Threshold value | >851 RLU |
| Sensitivity (%) | 80.77 |
| Specificity (%) | 90.00 |
| Test accuracy (%) | 83.33 |
| Positive predictive | 95.45 |
| value (%) | |
| Negative predictive | 64.29 |
| value (%) | |

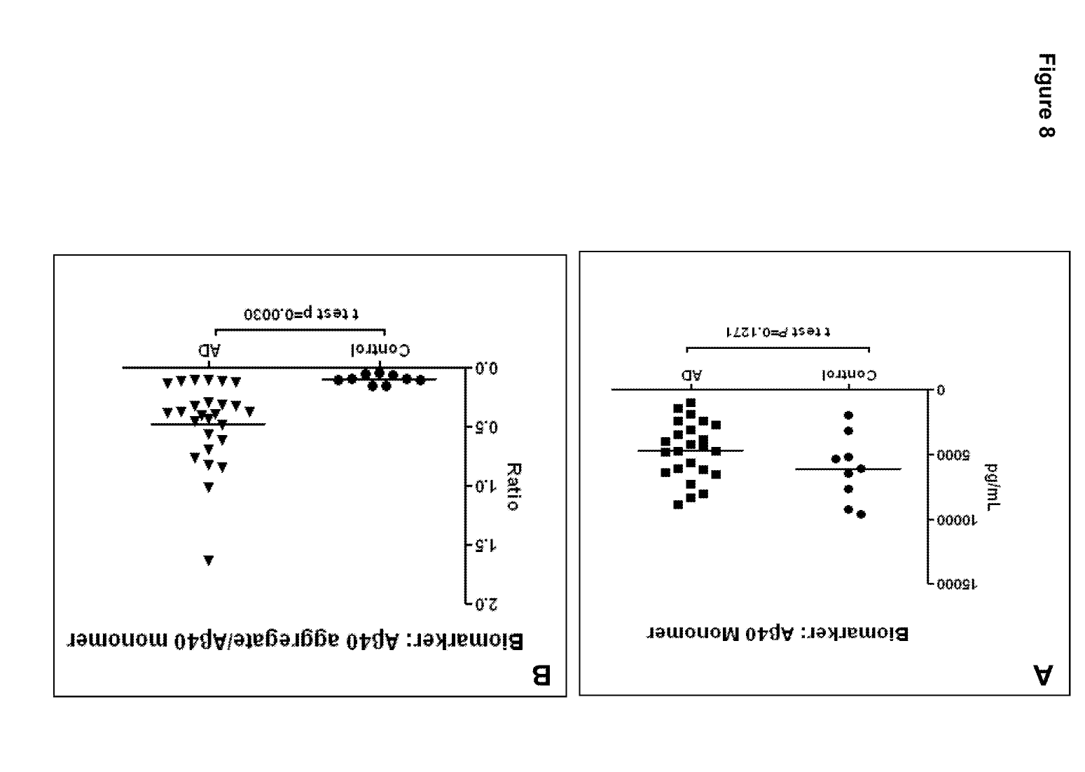

To assess whether Aβ40 aggregate could also be a useful biomarker for early stage AD only, additional ROC analyses were conducted to focus on these samples. The ROC curve is shown in FIG. 7. The diagnostic performance parameters are provided below.

| Parameters | Aβ40 Aggregate (MPA) |
|---|---|
| ROC AUC | 0.9538 |
| Threshold value | >861.0 RLU |
| Sensitivity (%) | 92.31 |
| Specificity (%) | 90.00 |
| Test accuracy (%) | 91.30 |
| Positive predictive | 92.31 |
| value (%) | |
| Negative predictive | 90.00 |
| value (%) | |

The MPA assay was also used to test the samples for Aβ42 aggregate levels. No statistically significant increase in Aβ42 aggregate signal was detected in any AD samples. (Data not shown.)

### Example 4

### The Ratio of Aβ40 Aggregate to Aβ42 Monomer is a Biomarker for Both AD and Early Stage AD

This Example shows a significant decrease of the Aβ42 monomer levels and a significant increase of the ratio of Aβ40 aggregate level to Aβ42 monomer level in CSF samples from patients with Alzheimer's disease.

To identify other biomarkers which may be useful either alone or in combination with Aβ40 aggregate, experiments were conducted to assess the level of Aβ42 monomer in the samples described in Example 3.

CSF samples were diluted 1:3 in assay buffer and applied and processed according to the protocol of the MSD® 96-Well MULTI-SPOT® Human/Rodent [4G8] Aβ Triplex Ultra-Sensitive Assay kit described in Example 1. This kit employs an Aβ42 antibody directed to the C-terminal tail region of Aβ, which is thought to be buried when Aβ42 is in aggregate form. Since the samples were not denatured as they were in Example 1, the vast majority of Aβ42 detected by this method is expected to be in monomeric form.

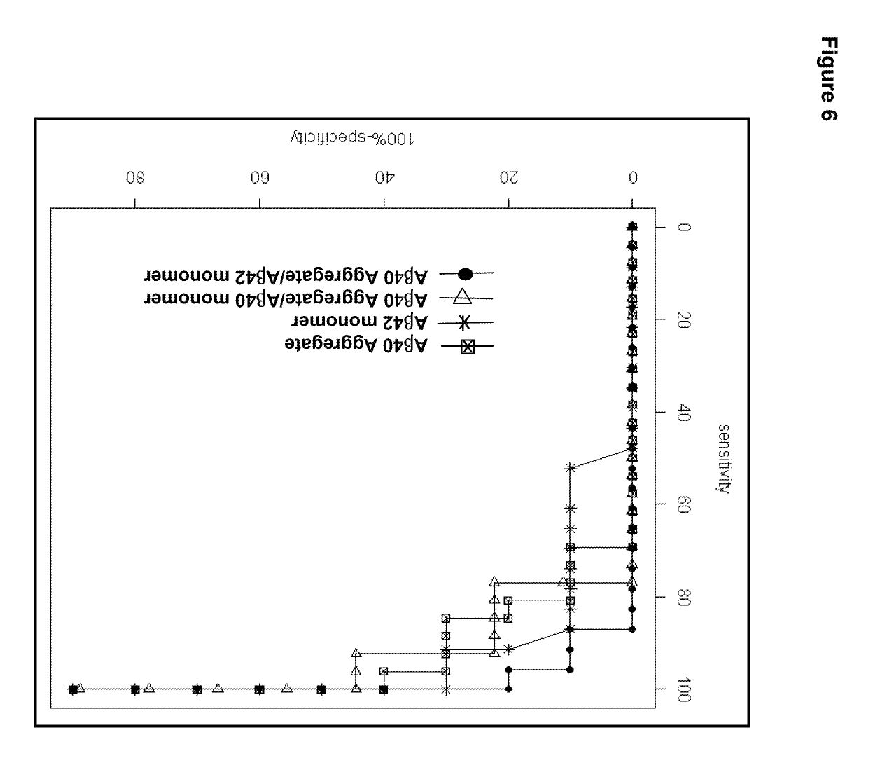

FIG. 5A shows a statistically significant decrease of the Aβ42 monomer levels in CSF samples from patients with AD.

The ratio of Aβ40 aggregate level to Aβ42 monomer level was calculated for each sample. FIG. 5B shows a statistically significant increase in the ratio for patients with AD.

ROC analysis was performed using Aβ42 monomer levels only or the ratio of Aβ40 aggregate level to Aβ42 monomer level. A ROC curve including the Aβ40 aggregate data provided in the previous Example is shown in FIG. 6. The diagnostic performance parameters for AD are provided below:

| | Aβ40 | Aβ42 | |
|---|---|---|---|
| | Aggregate | Monomer | Aβ40 Aggregate/ |
| Parameters | (MPA) | (ELISA) | Aβ42 Monomer |
| ROC AUC | 0.9307 | 0.93043 | 0.98261 |
| Threshold value | >851 RLU | <145 pg/ml | >4.33 |
| Sensitivity (%) | 80.77 | 86.96 | 95.65 |
| Specificity (%) | 90.00 | 90.00 | 90.00 |
| Test accuracy (%) | 83.33 | 87.88 | 93.94 |
| Positive predictive | 95.45 | 95.24 | 95.65 |
| value (%) | | | |
| Negative predictive | 64.29 | 75.00 | 90.00 |
| value (%) | | | |

All three measures gave very good sensitivity, specificity and accuracy values, although the ratio of Aβ40 aggregate level to Aβ42 monomer level produced the best among the three.

To assess whether this ratio could also be a useful biomarker for early stage AD only, the samples were also separated according to disease stage using the criteria described in Example 1 (FIG. 5C). T-tests show a significant difference between the early stage AD sample group and the control group, between the progressive substage AD sample group and the control group, and between the late substage AD sample group and the control group. A ROC curve including the Aβ40 aggregate data from the previous Example is shown in FIG. 7. The diagnostic performance parameters for early AD are provided below.

| | Aβ40 | | |
|---|---|---|---|
| | Aggregate | Aβ42 | Aβ40 Aggregate/ |
| Parameters | (MPA) | Monomer | Aβ42 Monomer |
| ROC AUC | 0.9538 | 0.9167 | 0.9750 |
| Threshold value | >861.0 RLU | <143.5 pg/mL | >4.995 |
| Sensitivity (%) | 92.31 | 83.33 | 91.67 |
| Specificity (%) | 90.00 | 90.00 | 90.00 |
| Test accuracy (%) | 91.30 | 86.36 | 90.91 |
| Positive predictive | 92.31 | 90.91 | 91.67 |
| value (%) | | | |
| Negative predictive | 90.00 | 81.82 | 90.00 |
| value (%) | | | |

### Example 5

### The Ratio of Aβ40 Aggregate to Aβ40 Monomer is Increased in CSF Samples from Alzheimer's Disease Patients

This Example shows a significant increase of the ratio of Aβ40 aggregate level to Aβ40 monomer level in CSF samples from patients with Alzheimer's disease and that this ratio is a useful biomarker for AD.

To identify additional biomarkers useful either alone or in combination with Aβ40 aggregate, experiments were conducted to assess the level of Aβ40 monomer in the samples described in Example 3.

CSF samples were diluted 1:11 in assay buffer and applied and processed to the protocol of the MSD® 96-Well MULTI-SPOT® Human/Rodent [4G8] Aβ Triplex Ultra-Sensitive Assay kit described in Example 1. This kit employs an Aβ40 antibody directed to the C-terminal tail region of Aβ40, which is thought to be buried when Aβ40 is in aggregate form. Since the samples were not denatured as they were in Example 1, the vast majority of Aβ40 detected by this method is expected to be in monomeric form.

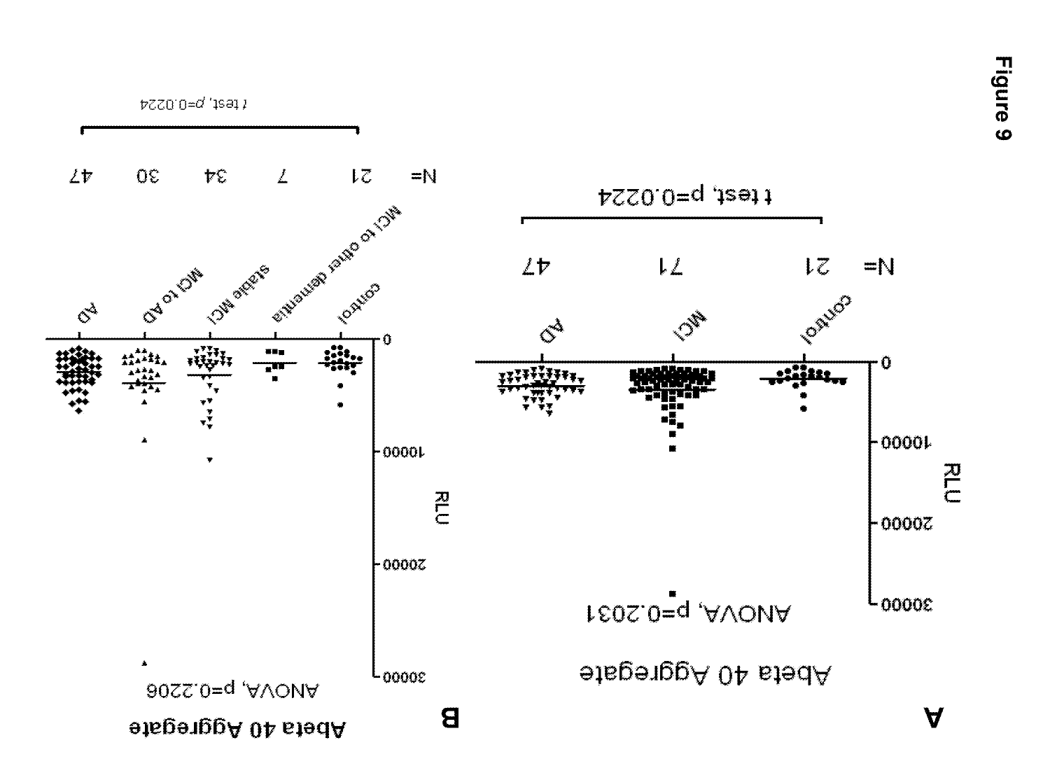

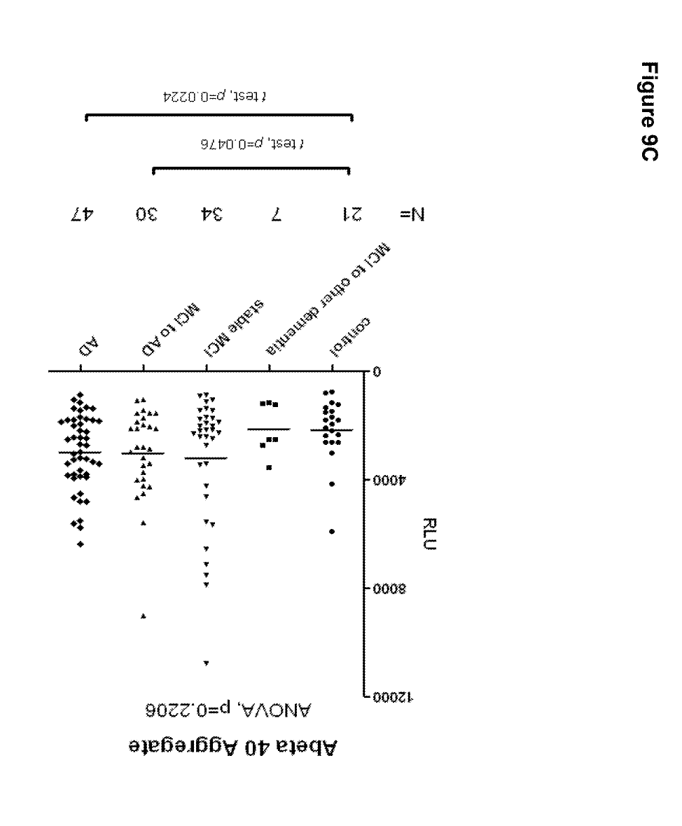

There is no significant change of the Aβ40 monomer levels in CSF samples from Alzheimer's disease patients (FIG. 8A). However, there is a statistically significant increase of ratio of Aβ40 aggregate level to Aβ40 monomer level as shown in FIG. 8B.

ROC analysis was performed using the ratio of Aβ40 aggregate level to Aβ40 monomer level and the ROC curve is shown in FIG. 6. The diagnostic performance parameters are as described below:

| Parameters | Ab40 Aggregate (MPA)/Ab40 Monomer |
|---|---|
| ROC AUC | 0.93162 |
| Threshold value | >0.111 |
| Sensitivity (%) | 92.31 |
| Specificity (%) | 77.78 |
| Test accuracy (%) | 88.57 |
| Positive predictive | 92.31 |
| value (%) | |
| Negative predictive | 77.78 |
| value (%) | |

The ratio of Aβ40 aggregate level to Aβ40 monomer level has good sensitivity, specificity, and accuracy characteristics.

### Example 6

### A Larger Collection of Clinical CSF Samples Confirms that Aβ40 Aggregate Levels are Increased in Both Mild Cognitive Impairment Patients and Alzheimer's Disease Patients and that Aβ40 Aggregate is a Biomarker for Both AD and a Subset of MCI Patients Who Progress to AD

This Example shows that elevated levels of Aβ40 aggregates can be detected in a large set of CSF samples from both MCI and AD patients and that Aβ40 aggregate level is a useful biomarker for AD and early stage AD comprised of MCI patients whom progress to a clinical diagnosis of AD.

This larger set of CSF samples was obtained from a university research hospital. The AD group at the time of CSF collection consisted of 47 patients consulting the memory disorder clinic, mean age 75.7±6.6 years. Patients diagnosed with AD had to meet the DSM-IIIR criteria of dementia [American Psychiatric Association (1987) Diagnostic and Statistical Manual of Mental Disorders, Third edition revised. American Psychiatric Association, Washington, D.C., USA] and the criteria of probable AD defined by NINCDS-ADRDA [McKann G, Drachman D, Folsyein M, Katzman R, Pricxe D, Stadlan E M (1984) Clinical diagnosis of Alzheimer's disease: report of the NINCDS-ARADA Work Group under the auspices of Department of Health Service Task Force on Alzheimer's Disease. Neurology 34, 939-944]. The AD patients were followed over time with repeated clinical evaluations, which increases the clinical diagnostic accuracy. The MCI group at the time of CSF collection consisted of 71 patients, mean age 72.9±7.7 years and had to meet the criteria for MCI advocated by Petersen [Petersen R C (2004) Mild cognitive impairment as a diagnostic entity. J Intern Med 256, 183-194] and included subjective memory complaint, objective memory impairment confirmed by a physician, preservation of general cognitive functioning with a MMSE score of ≧24, minimal impairment of daily life activities, and failure to meet the DSM-IIIR criteria of dementia. The control group consisted of healthy elderly volunteers, mean age 76.6±6.5 years, with absence of memory complaints or any other cognitive symptoms, and no signs of active neurological or psychiatric disease.

MCI patients were clinically followed for a mean of 4.7 years for subsequent conversion to AD or other types of dementia. A subsequent diagnosis of AD had to meet the same criteria for AD as described previously for the AD group at the time of CSF collection. CSF was collected in polypropylene tubes, centrifuged, aliquoted, and stored at −80° C. pending analyses.

100 μl of each of these CSF samples was incubated with 25 μl of 5×TBSTT (250 mM Tris, pH 8.4; 750 mM NaCl; 4.15% Tween-20; 4.15% Triton X-100) and 30 μl of PSR1 (peptoid reagent XIIb covalently bound to Dynal M270-carboxylic acid beads at 30 mg/mL) at 37° C. for 1 hour with shaking at 500 rpm.

A negative control for the assay was carried out using 100 μl/assay of normal human CSF and 25 μl of 5×TBSTT buffer in triplicate. A positive control was carried out using 100 μl positive control CSF (CSF from individuals with diagnosed Alzheimer's disease) and 25 μl of 1×TBSTT.

The beads were washed eight times with TBST (50 mM Tris, pH 7.5; 150 mM NaCl; 0.05% Tween-20) using magnetic separation to immobilize the beads while the wash buffer was removed. 100 μl of 1% Zwittergent 3-14 (from Sigma, product number T7763) was added and the mixture was incubated at room temperature for 30 minutes with shaking at 750 rpm. The beads were washed again 8 times with TBST using magnetic separation. Aggregates were eluted and denatured by adding 20 μl of 0.15 N NaOH and incubating at room temperature for 30 minutes with shaking at 750 rpm. The assay plate was immediately neutralized with the addition of 20 μl of 0.18 M NaH2PO4 containing 0.4% Tween-20. The mixture was incubated for 5 minutes at room temperature with shaking at 750 rpm. Magnetic separation was applied to the assay plate and the supernatants were transferred to an ELISA plate which is a component of a kit from Meso Scale Discovery (Gaithersburg, Md.), the MSD® 96-Well MULTI-SPOT® Human/Rodent [4G8] Abeta Triplex Ultra-Sensitive Assay. ELISA was carried out according to the protocol of the kit which allows for multiplex detection of Aβ38, Aβ40 and Aβ42. In this Example, 47 AD, 71 MCI and 21 control samples were grouped into 3 different populations based on clinical diagnosis. The MCI samples were then further subdivided based on follow up clinical diagnosis made after the CSF sample collection.

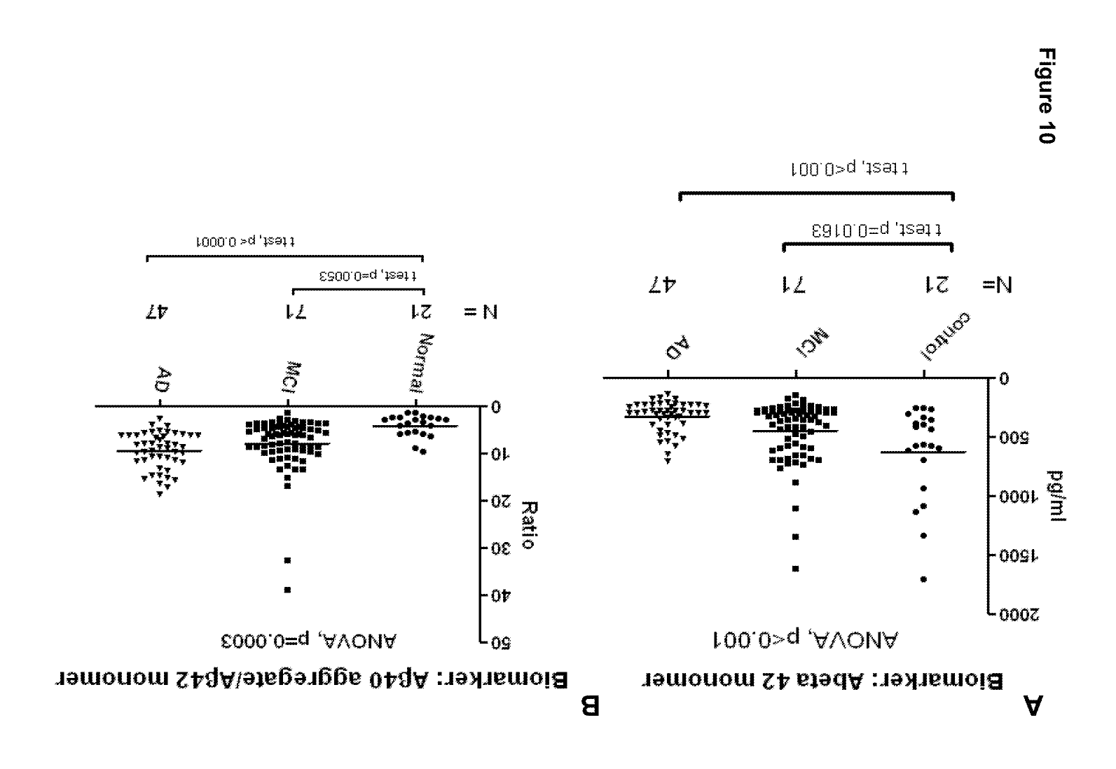

FIG. 9A confirms using a another larger set of CSF samples that Alzheimer's disease patients have higher levels of Aβ40 aggregates than cognitively normal control samples. In FIG. 9B the MCI patient samples have been further divided into 7 MCI patients whom later progressed to other, non-AD type dementia, 34 stable MCI patients, and 30 MCI patients who progressed onto clinical diagnosed AD. There is a statistical significant increase in Ab40 aggregate level in the AD. In FIG. 9C removal of a high signal outlier in the MCI to AD group provides additional statistical significance between the control and MCI to AD groups

To confirm that Aβ42 monomer may be useful either alone or in combination with Aβ40 aggregate, experiments were also conducted to assess the level of Aβ42 monomer in the samples. CSF samples were diluted 1:3 in assay buffer and applied and processed according to the protocol of the MSD® 96-Well MULTI-SPOT® Human/Rodent [4G8] Aβ Triplex Ultra-Sensitive Assay kit described in Example 1. This kit employs an Aβ42 antibody directed to the C-terminal tail region of Aβ, which is thought to be buried when Aβ42 is in aggregate form. Since the samples were not denatured as they were in Example 1, the vast majority of Aβ42 detected by this method is expected to be in monomeric form.

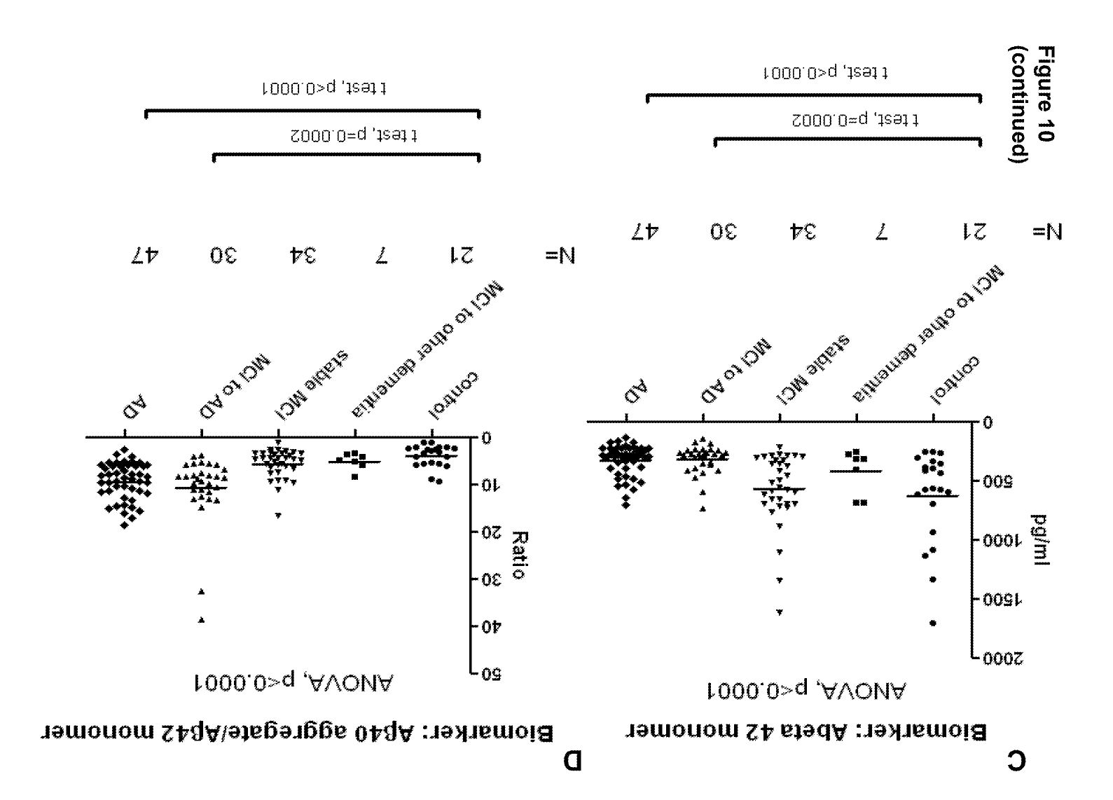

FIG. 10A shows a statistically significant decrease of the Aβ42 monomer levels in CSF samples from patients with either MCI or AD relative to matched cognitive normal controls. The ratio of Aβ40 aggregate level to Aβ42 monomer level was also calculated for each sample. FIG. 10B shows a statistically significant increase in the ratio for patients with either MCI or AD. Similar Aβ42 monomer level values and ratios of Aβ40 aggregate level to Aβ42 monomer level are also shown for the MCI subsets in FIGS. 10C and 10D respectively. FIG. 10D shows a statistically significant increase in the ratio for both AD patients with MCI patients who later progressed to AD.

ROC analysis was conducted to determine the optimal sensitivity and specificity for Alzheimer's disease. Prism version 5.0 for Windows (GraphPad Software; La Jolla, Calif.) was used to conduct the ROC analysis. This program generates an ROC curve and the associated AUC, Sensitivity, and Specificity values for a number of different threshold/cutoff values. The optimal threshold/cutoff value which provides for the best sensitivity and specificity is then manually determined by simply choosing the threshold value which provides maximal sensitivity and specificity (i.e. the smallest difference between these two parameters).

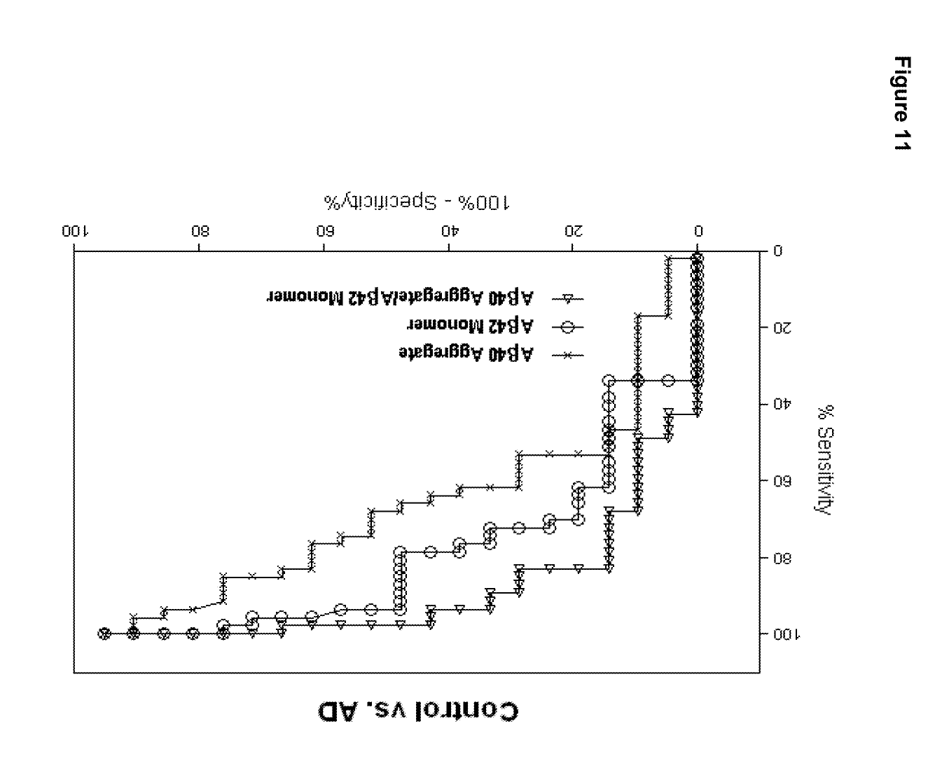

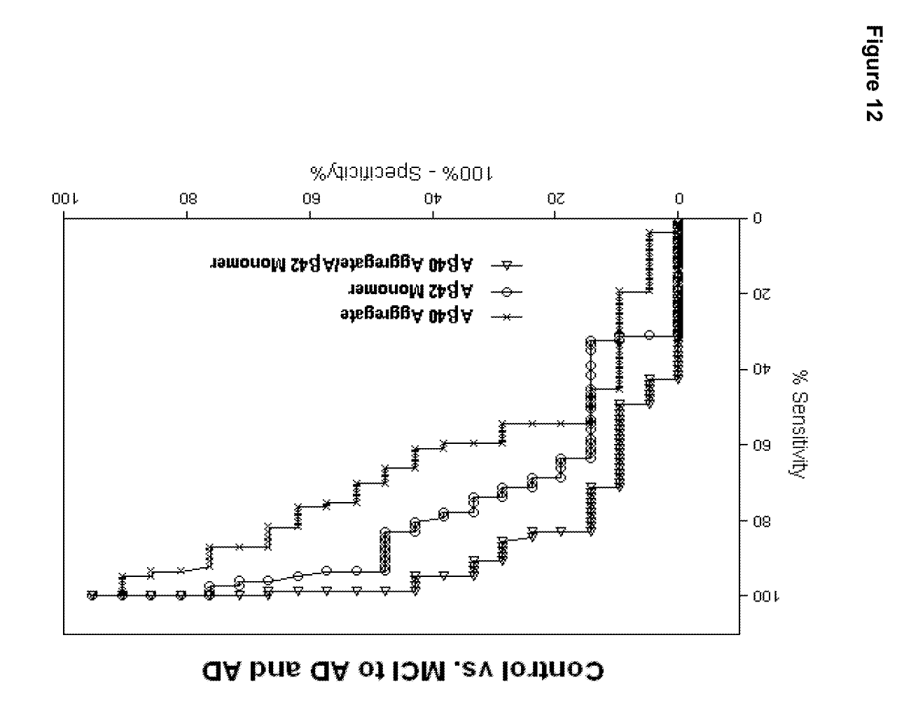

The ROC curve for diagnosis of AD is shown in FIG. 11. The diagnostic performance parameters for Aβ40 aggregate, Aβ42 monomer, and ratio of Aβ40 aggregate level to Aβ42 monomer level are provided below.

| | Aβ40 | | |
|---|---|---|---|
| | Aggregate | Aβ42 | Aβ40 Aggregate/ |
| Parameters | (MPA) | Monomer | Aβ42 Monomer |
| ROC AUC | 0.6824 | 0.8019 | 0.8926 |
| Threshold value | >2441 | <344.5 | >5.980 |
| Sensitivity (%) | 61.70 | 70.21 | 82.98 |
| Specificity (%) | 71.43 | 76.19 | 85.71 |
| Test accuracy (%) | 64.71 | 72.06 | 83.82 |
| Positive predictive | 82.86 | 86.84 | 92.86 |
| value (%) | | | |
| Negative predictive | 45.45 | 53.33 | 69.23 |
| value (%) | | | |

All three measures gave very good sensitivity, specificity and accuracy values, although the ratio of Aβ40 aggregate level to Aβ42 monomer level produced the best among the three.

### Aβ40 Aggregate as a Biomarker for AD

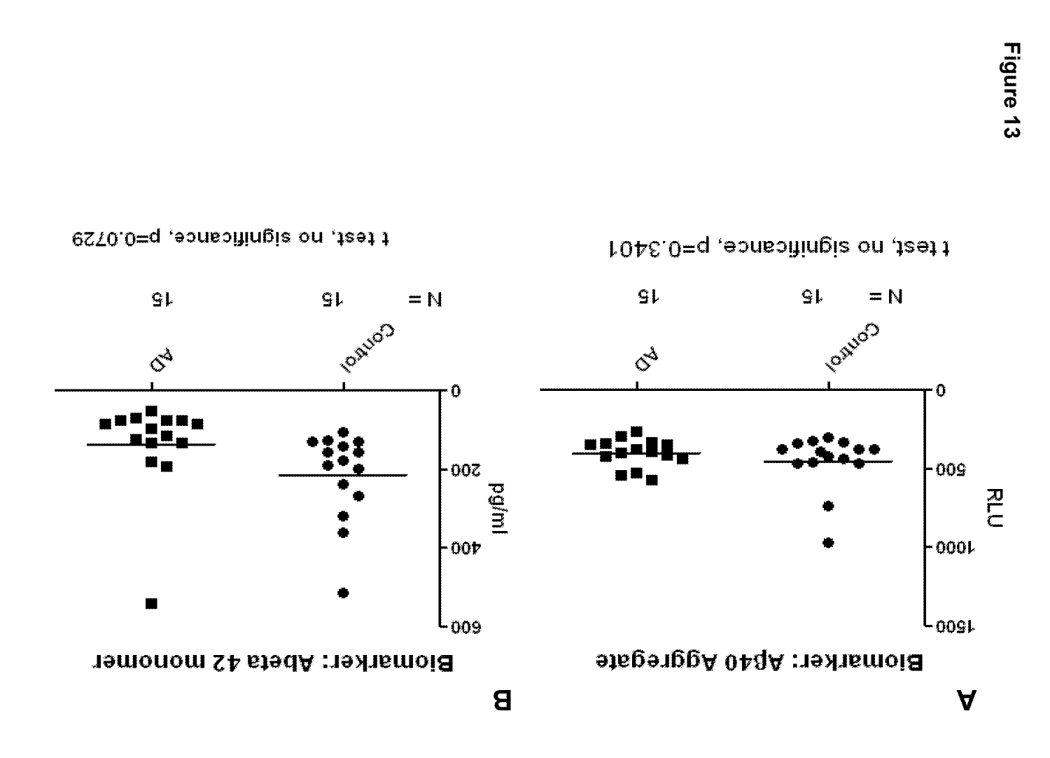

To assess whether Aβ40 aggregate could also be a useful biomarker for detection of AD as defined by specific detection of the MCI patients who progressed to AD and AD patients, additional ROC analyses of all data points in FIG. 9B were conducted to focus on these samples. The ROC curve is shown in FIG. 12. The threshold value shown is for both MCI to AD conversion and AD. The diagnostic performance parameters are provided below.

| | Aβ40 | | |
|---|---|---|---|
| | Aggregate | Aβ42 | Aβ40 Aggregate/ |
| Parameters | (MPA) | Monomer | Aβ42 Monomer |
| ROC AUC | 0.6849 | 0.8018 | 0.8995 |
| Threshold value | >2441 | <340.5 | >5.975 |
| Sensitivity (%) | 59.74 | 68.83 | 83.12 |
| Specificity (%) | 71.43 | 80.95 | 85.71 |
| Test accuracy (%) | 62.24 | 71.43 | 83.67 |
| Positive predictive | 88.46 | 92.98 | 95.52 |
| value (%) | | | |
| Negative predictive | 32.61 | 41.46 | 58.06 |
| value (%) | | | |

All three measures gave very good sensitivity, specificity and accuracy values, although the ratio of Aβ40 aggregate level to Aβ42 monomer level produced the best among the three.

The MPA assay was also used to test the samples for Aβ42 aggregate levels. No statistically significant increase in Aβ42 aggregate signal was detected in any AD samples. (Data not shown).

### Aβ40 Aggregate as a Biomarker for MCI Progressing to AD

To assess whether Aβ40 aggregate could also be a useful biomarker for MCI patients who later progress to AD, the three threshold values for detection of AD generated in the ROC analysis summarized in FIG. 11 and the corresponding table above were applied to the samples from the subset of MCI patients that later progressed to AD or other types of non-AD dementia. The diagnostic utility of these threshold values for Aβ40 aggregate, Aβ42 monomer, and Aβ40 Aggregate/Aβ42 Monomer ratio to predict subsequent progression to AD is summarized below.

| | Aβ40 | | |
|---|---|---|---|
| | Aggregate | Aβ42 | Aβ40 Aggregate/ |
| Parameters | (MPA) | Monomer | Aβ42 Monomer |
| Threshold value | >2441 | <344.5 | >5.980 |
| Sensitivity (%) | 56.67 | 66.67 | 83.33 |
| Specificity (%) | 42.86 | 47.86 | 71.43 |
| Test accuracy (%) | 54.05 | 62.16 | 81.08 |
| Positive predictive | 80.95 | 83.33 | 92.60 |
| value (%) | | | |
| Negative predictive | 18.75 | 23.08 | 50.00 |
| value (%) | | | |

All three measures gave very good sensitivity, specificity and accuracy values, although the ratio of Aβ40 aggregate level to Aβ42 monomer level produced the best among the three.

### Example 7

### Another Collection of Clinical CSF does not Confirm that Aβ40 Aggregate Levels are Increased in Alzheimer's Disease Patients

This Example fails to show that elevated levels of Aβ40 aggregates can be detected in another independent set of CSF samples from AD patients and that Aβ40 aggregate level is a useful biomarker for AD

This set of CSF samples was obtained from the same university research hospital as the CSF samples evaluated in Example 6. Patients were diagnosed with the same criteria and collected in the same manner as discussed in Example 6.

200 μl of each of these CSF samples was incubated with 50 μl of 5×TBSTT (250 mM Tris, pH 7.5; 750 mM NaCl; 5% Tween-20; 5% Triton X-100) and 30 μl of PSR1 (peptoid reagent XIIb covalently bound to Dynal M270-carboxylic acid beads at 30 mg/mL) at 37° C. for 1 hour with shaking at 500 rpm.

A negative control for the assay was carried out using 200 μl/assay of normal human CSF and 50 μl of 5×TBSTT buffer (250 mM Tris, pH 7.5; 750 mM NaCl; 5% Tween-20; 5% Triton X-100) in triplicate. A positive control was carried out using 200 μl positive control CSF (CSF from individuals with diagnosed Alzheimer's disease) and 50 μl of 1×TBSTT.

The beads were washed eight times with TBST (50 mM Tris, pH 7.5; 150 mM NaCl; 0.05% Tween-20) using magnetic separation to immobilize the beads while the wash buffer was removed. 100 μl of 1% Zwittergent 3-14 (from Sigma, product number T7763) was added and the mixture was incubated at RT for 30 minutes with shaking at 750 rpm. The beads were washed again 8 times with TBST using magnetic separation. Aggregates were eluted and denatured by adding 20 μl of 0.1 N NaOH and incubating at 80° C. for 30 minutes with shaking at 750 rpm. The assay plate was immediately put on ice for one minute, centrifuged at 200×g for 1 min at 4° C., then neutralized with the addition of 20 μl of 0.12 M NaH2PO4 containing 0.4% Tween-20. The mixture was incubated for 5 minutes at room temperature with shaking at 750 rpm. Magnetic separation was applied to the assay plate and the supernatants were transferred to an ELISA plate which is a component of a kit from Meso Scale Discovery (Gaithersburg, Md.), the MSD® 96-Well MULTI-SPOT® Human/Rodent [4G8] Abeta Triplex Ultra-Sensitive Assay. ELISA was carried out according to the protocol of the kit which allows for multiplex detection of Aβ38, Aβ40 and Aβ42.

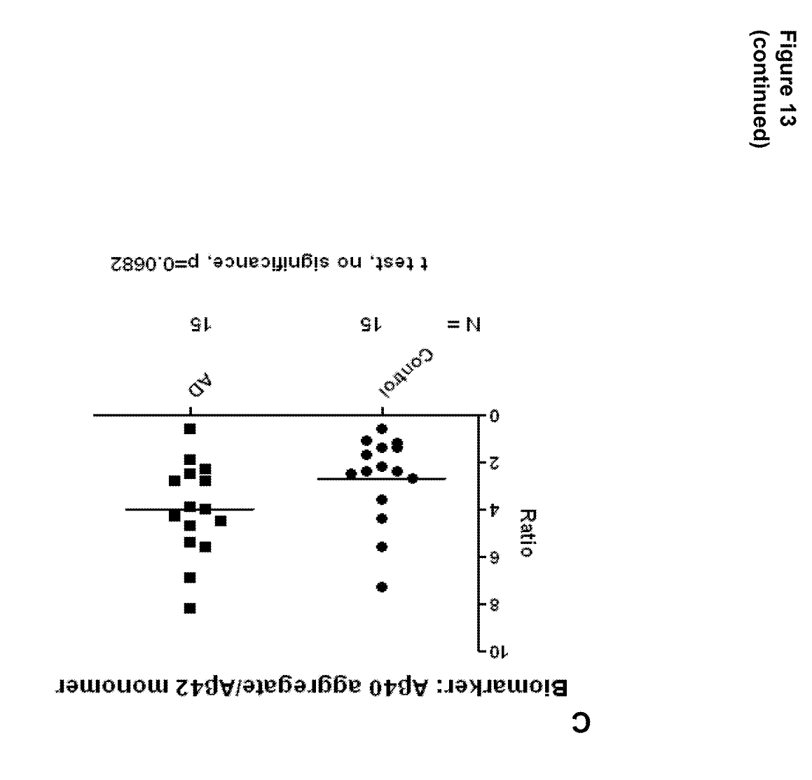

In this Example, 15 AD and 15 control samples were grouped into 2 different populations based on clinical diagnosis. FIGS. 13A, B and C show that no statistically significant change in either Aβ40 aggregate levels, Aβ42 monomer levels, or the ratio of Aβ40 aggregate level to Aβ42 monomer level was observed between CSF samples from cognitive normal controls or AD patients. We observed that in this example, in which a relatively small sample set was used, the levels of Aβ40 aggregate and Aβ42 monomer detected are much lower than the levels detected in the previous examples. The small sample set and the low detection levels may explain why this example cannot confirm that elevated levels of Aβ40 aggregates can be detected in CSF samples from AD.

### Example 8

### Sizing Aβ40 Aggregates from AD CSF by Differential Centrifugation

This Example characterizes the physical properties of the aggregates captured from Alzheimer's Disease CSF by PSR1.

AD CSF or normal pooled CSF spiked with nothing, 5 ng/mL globulomer or 200 mL/mL ADBH (with or without sonication) were centrifuged at 16,000×g for 10 min or 134,000×g for 1 hour at 4 C. Supernatant and pellet fractions were taken to separate tubes (pellets were reconstituted in CSF with the same volume as the original sample) and subjected to the Misfolded Protein Assay (MPA).

Misfolded Protein Assay: 100 ul sample was incubated with 25 ul 5×TBSTT buffer (250 mM Tris, 750 mM NaCl, 5% Tween20, 5% TritonX-100 pH 7.5) and 30 ul PSR1 beads for 1 hour at 37 C. Beads were washed 6× with TBST followed by a 30 minute incubation with 1% Zwittergent 3-14 and another TBST wash. Abeta peptide was eluted with 0.15 M NaOH for 30 minutes at room temperature, followed by neutralization of the eluate with 0.18 M NaH2PO4+0.5% Tween20 and detection by Mesoscale's triplex Aβ immunoassay according to manufacturer's instructions.

The behavior of the various aggregates of known sizes was determined to provide molecular weight references for the aggregates found in Alzheimer's CSF. Although the solubility of aggregates does not necessarily have a linear relationship with molecular weight (and is subject to variability depending on the conformation of the aggregates), these studies provide some frame of reference for aggregate size. Abeta fibrils from an unsonicated Alzheimer's Disease Brain Homogenate (ADBH) pelleted at both 16,000 g and 134,000 g. These aggregates eluted near the void volume of a TSK4000 column and are likely to be greater than 1 MDa. Some proportion of Abeta aggregates from a sonicated ADBH was soluble at 16,000 g but pelleted at 134,000 g. Size exclusion chromatography estimated these aggregates to be about 0.5-1 MDa. Globulomers (estimated to be approximately 54 KDa) were soluble at both 16,000 g and 134,000 g.

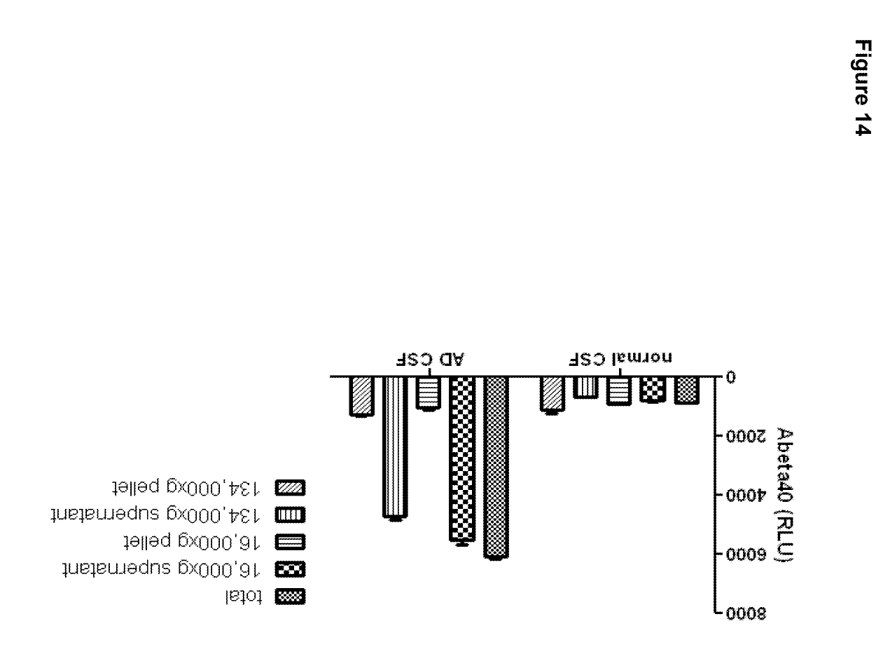

FIG. 14 depicts the amount of Aβ40 aggregates detected by the Misfolded Protein Assay in the supernatant and pellet of Alzheimer's Disease CSF and normal CSF centrifuged at 16,000 g or 134,000 g. Endogenous Aβ40 aggregates in AD CSF stay in solution after a 1 hour centrifugation at 134,000 g, suggesting that they may be smaller than “intermediate-sized” aggregates of 0.5 to 1 MDa found in a sonicated ADBH sample. These data indicate that the oligomers found in AD CSF have different behavior with respect to solubility when compared to aggregates deposited in tissues (ADBH) and is suggestive that they are smaller in size.

1. A method for assessing increased probability of having Alzheimer's disease in a subject under assessment comprising a step of obtaining a measurement of Aβ40 aggregates in a biological sample from said subject, wherein said biological sample does not comprise brain tissue, a fraction of brain tissue or brain homogenate.

2. A method for assessing increased probability of MCI progressing to Alzheimer's disease in a subject under assessment comprising a step of obtaining a measurement of Aβ40 aggregates in a biological sample from said subject, wherein said biological sample does not comprise brain tissue, a fraction of brain tissue or brain homogenate.

3. The method of claim 1 or 2, further comprising a step of reporting said Aβ40 aggregate measurement to a reporting means comprising a visual display or a printer.

4. The method of claim 1 or 2 wherein said Aβ40 aggregate measurement comprises the level of Aβ40 aggregates in said biological sample.

5. The method of claim 1 or 2, wherein said biological sample from said subject under assessment comprises bodily fluid or bodily tissue.

6. The method of claim 5, wherein said biological sample comprises a member selected from the group consisting of: whole blood, blood fractions, blood components, plasma, platelets, serum, cerebrospinal fluid (CSF), bone marrow, urine, tears, milk, lymph fluid, organ tissue, nervous system tissue, non-nervous system tissue, muscle tissue, biopsy, necropsy, fat biopsy, fat tissue, cells, feces, placenta, spleen tissue, lymph tissue, pancreatic tissue, bronchoalveolar lavage (BAL), or synovial fluid.

7. The method of claim 6, wherein said biological sample comprises a member selected from the group consisting of: plasma, serum, CSF, and urine.

8. The method of claim 5, wherein said Aβ40 aggregates are circulating.

9. The method of claim 1 or 2, wherein said biological sample comprises bodily tissue; and wherein said Aβ40 aggregate measurement is obtained by a method comprising the steps of:
providing a homogenate of said bodily tissue;
contacting said homogenate with an aggregate-specific binding reagent under conditions that allow binding of said reagent to Aβ40 aggregates, if present, to form a complex; and
detecting Aβ40 aggregates, if any, in said subject biological sample by its binding to said reagent; wherein said reagent is attached to a solid support and binds preferentially to aggregate over monomer when attached to said solid support.

10. The method of claim 4, further comprising a step of determining that said subject has an increased probability of having Alzheimer's disease or MCI progressing to Alzheimer's disease if said Aβ40 aggregate level is higher than a control level threshold,
wherein said control level threshold is calculated from data comprising the levels of Aβ40 aggregates in biological samples from a plurality of cognitively normal control subjects;
wherein said subject under assessment and said control subjects are of a same species; and
wherein all of said biological samples comprise CSF.

11. The method of claim 1 or 2, wherein said Alzheimer's disease is in early stage.

12. A method for assessing increased probability of not having Alzheimer's disease in a subject under assessment comprising the steps of:
obtaining the level of Aβ40 aggregates in a biological sample from said subject; and
determining that said subject has an increased probability of not having Alzheimer's disease if said subject does not show clinical cognitive impairment and if said Aβ40 aggregate level is the same as or lower than a control level threshold;
wherein said control level threshold is calculated from data comprising the levels of Aβ40 aggregates in biological samples from a plurality of cognitively normal control subjects;
wherein said subject under assessment and said control subjects are of a same species; and
wherein all of said biological samples comprise CSF and none of said biological samples comprises brain tissue, a fraction of brain tissue or brain homogenate.

13. A method for assisting in assessing increased probability of not having Alzheimer's disease in a subject under assessment comprising the steps of:
measuring the level of Aβ40 aggregates in a biological sample from said subject; and
communicating said Aβ40 aggregate level to a different entity;
wherein said different entity determines that said subject has an increased probability of not having Alzheimer's disease if said subject does not show clinical cognitive impairment and if said Aβ40 aggregate level is the same as or lower than a control level threshold;
wherein said control level threshold is calculated from data comprising the levels of Aβ40 aggregates in biological samples from a plurality of cognitively normal control subjects;
wherein said subject under assessment and said control subjects are of a same species; and
wherein all of said biological samples comprise CSF and none of said biological samples comprises brain tissue, a fraction of brain tissue or brain homogenate.

14-44. (canceled)

45. A method for assessing increased probability of MCI progressing to Alzheimer's disease in a subject under assessment comprising the steps of:
obtaining the level of Aβ40 aggregates in a biological sample from said subject, determining that said subject has an increased probability of MCI progressing to Alzheimer's disease if said Aβ40 aggregate level is higher than a control level threshold,
wherein said control level threshold is calculated from data comprising the levels of Aβ40 aggregates in biological samples from a plurality of cognitively normal control subjects;
wherein said subject under assessment and said control subjects are of a same species; and
wherein all of said biological samples comprise CSF, and wherein none of said biological samples comprise brain tissue, a fraction of brain tissue or brain homogenate.

46. A method for assisting in assessing increased probability of MCI progressing to Alzheimer's disease in a subject under assessment comprising the steps of:
measuring the level of Aβ40 aggregates in a biological sample from said subject; and
communicating said Aβ40 aggregate level to a different entity;
wherein said different entity determines that said subject has an increased probability of MCI progressing to Alzheimer's disease if said Aβ40 aggregate level is higher than a control level threshold;
wherein said control level threshold is calculated from data comprising the levels of Aβ40 aggregates in biological samples from a plurality of cognitively normal control subjects;
wherein said subject under assessment and said control subjects are of a same species; and
wherein all of said biological samples comprise CSF and none of said biological samples comprises brain tissue, a fraction of brain tissue or brain homogenate.

47. (canceled)

48. (canceled)

49. The method of claim 1 or 2 wherein said subject under assessment is a human.

50. (canceled)

51. The method of claim 49, wherein said subject under assessment is alive.

52. (canceled)

53. The method of claim 10, wherein said biological sample is collected using a same method in the same manner as said biological samples from said control subjects.

54. The method of claim 10, wherein said Aβ40 aggregate level is obtained by a method comprising the steps of:
contacting said CSF with an aggregate-specific binding reagent under conditions that allow binding of said reagent to Aβ40 aggregates, if present, to form a complex; and
detecting Aβ40 aggregates, if any, in said subject CSF by its binding to said reagent;
wherein said reagent is attached to a solid support and binds preferentially to aggregate over monomer when attached to said solid support.

55. The method of claim 54, wherein said detecting step comprises the substeps of:
separating said complex formed by said reagent and Aβ40 aggregates from unbound monomers of Aβ40 if present;
optionally, dissociating Aβ40 aggregates from said complex; and
detecting Aβ40 aggregates.

56. The method of claim 54, wherein said detecting step comprises the substeps of:
separating said complex formed by said reagent and Aβ40 aggregates from unbound monomers of Aβ40, if present, and removing said unbound monomers of Aβ40;
denaturing said Aβ40 aggregates present in said complex to form Aβ40 monomers; and
detecting Aβ40 monomers.

57. (canceled)

58. The method of claim 54, wherein said reagent comprises a member selected from the group consisting of: peptoid, peptide, and dendron.

59. (canceled)

60. (canceled)

61. The method of claim 54, wherein said solid support is selected from the group consisting of: nitrocellulose, polystyrene latex, polyvinyl fluoride, diazotized paper, nylon membrane, activated bead, magnetically responsive bead, titanium oxide, silicon oxide, polysaccharide bead, polysaccharide membrane, agarose, glass, polyacrylic acid, polyethyleneglycol, polyethyleneglycol-polystyrene hybrid, controlled pore glass, glass slide, gold bead, and cellulose.

62. The method of claim 54, wherein said reagent is detectably labeled.

63-64. (canceled)

65. The method of claim 1, further comprising a step of obtaining a measurement of a second indicator of Alzheimer's disease in said subject under assessment.

66. The method of claim 2, further comprising a step of obtaining a measurement of a second indicator of progression of MCI to Alzheimer's disease in said subject under assessment.

67. The method of claim 65 or 66, wherein said second indicator is Aβ42 monomer.

68. The method of claim 67, wherein all of said biological samples comprise CSF, said method further comprising the steps of:
calculating a ratio of Aβ40 aggregate level to Aβ42 monomer level in said subject under assessment;
comparing said ratio to a control ratio threshold which is calculated from data comprising the levels of Aβ40 aggregates and the levels of Aβ42 monomers in biological samples from a plurality of cognitively normal control subjects; and
determining that said subject under assessment has an increased probability of having Alzheimer's disease or MCI progressing to Alzheimer's disease if said ratio is higher than said control ratio threshold.
